(19) United States
(12) Patent Application Publication
(10) Pub. No.: US 2011/0282031 A1
(43) Pub. Date: Nov. 17, 2011

(54) A1 ADENOSINE RECEPTOR DIAGNOSTIC PROBES

(76) Inventors: Constance N. Wilson, Raleigh, NC (US); John J. Partridge, Chapel Hill, NC (US)

(21) Appl. No.: 13/010,152

(22) Filed: Jan. 20, 2011

Related U.S. Application Data
(63) Continuation-in-part of application No. 11/754,077, filed on May 25, 2007, now Pat. No. 7,902,360.
Application No. 11/754,077 is a continuation of application No. 10/861,677, filed on Jun. 4, 2004, now Pat. No. 7,247,639.
(60) Provisional application No. 60/476,684, filed on Jun. 6, 2003.

(51) Int. Cl. C07D 239/545; C07F 19/00; C07K 14/435; C07D 473/06

(52) U.S. Cl. 530/350; 544/311; 544/272; 544/270; 544/268; 534/15; 534/10

(57) ABSTRACT

This invention relates to a diagnostic probe comprising a compound of formula (I):

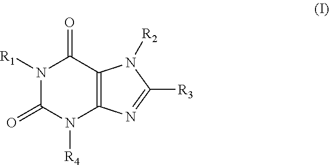

such as 3-[2-[4-(5-aminopentyl-3-amidocarboxypropanoyl)]aminophenyl]ethyl]-8-benzyl-7-[2-ethyl(2-hydroxyethyl)amino]ethyl-1-propylxanthine, with one or more label moieties attached thereto. The compounds of the present invention are useful as diagnostic probes, including probes for diagnostic assays and imaging. Accordingly, the invention provides A₁ adenosine receptor antagonist compounds with radioactive or non-radioactive labels suitable for executing such assays and imaging measurements. Labeled compounds are useful to obtain quantitative measurements of the A₁ adenosine receptor antagonist compounds. They may be employed as an imaging agent in diagnostic procedures such as MRI and PET as well as cell or receptor based assays.

### RELATED APPLICATIONS

This application is a continuation-in-part of U.S. patent application Ser. No. 11/754,077 filed on May 25, 2007 which is a continuation of patent application Ser. No. 10/861,677 (issued as a patent on Jul. 24, 2007, U.S. Pat. No. 7,247,639) which claims priority of U.S. Provisional Patent Application Ser. No. 60/476,684 filed Jun. 6, 2003 and all are incorporated by reference herein in their entirety.

### FIELD OF THE INVENTION

The present invention concerns compounds useful as diagnostic probes for A₁ adenosine receptors.

### BACKGROUND OF THE INVENTION

Adenosine receptors are involved in a vast number of peripheral and central regulatory mechanisms such as, for example, vasodilation, cardiac depression, inhibition of lipolysis, inhibition of insulin release and potentiation of glucagon release in the pancreas, and inhibition of neurotransmitter release from nerve endings.

In general, adenosine receptors can be divided into two main classes, A₁ receptors which can inhibit, and A₂ receptors which can stimulate adenylate cyclase activity. One of the best known classes of adenosine receptor antagonists are the xanthines which include caffeine and theophylline. See e.g., Müller et al., J. Med. Chem. 33: 2822-2828 (1990).

In general, many of these antagonists often exhibit poor water solubility, and low potency or lack of selectivity for adenosine receptors. Additionally, selective analogues of adenosine receptor antagonists have been developed through the “functionalized congener” approach. Analogues of adenosine receptor ligands bearing functionalized chains have been synthesized and attached covalently to various organic moieties such as amines and peptides. Attachment of the polar groups to xanthine congeners has been found to increase water solubility. Nonetheless, such developments have yet to fully address problems associated with potency and selectivity.

### SUMMARY OF THE INVENTION

In one aspect, the invention is a diagnostic probe comprising a compound of formula (I):

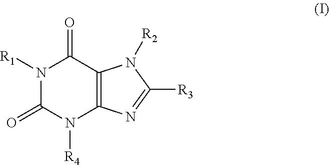

wherein;
R₁ is C₁₋₈ straight or branched alkyl optionally substituted with one or more OR₅, NR₆R₇, or halogen groups,
   wherein;
      R₅ and R₆ are independently H, or C₁₋₈ straight or branched alkyl;
      R₇ is H, C₁₋₈ straight or branched alkyl, or Alk₁-OH,
         wherein; Alk₁ is C₁₋₈ straight or branched alkylene;
R₂ is H, C₁₋₈ alkyl, Alk₂COOH, Alk₃COOR₈, Alk₄CONR₉R₁₀, Alk₅OH, Alk₆SO₃H, Alk₇PO₃H₂, Alk₈OR₁₁, Alk₉OH or Alk₁₀NR₁₂R₁₃, or, when R₃ is (CH₂)_{q}(C₆H₄)Q, R₂ is as defined above or is Alk₁₁N(CH₃)Alk₁₂OH; and when R₃ is other than (CH₂)_{q}(C₆H₄)Q, R₂ is as defined above or is Alk₁₃NR₁₄R₁₅;
   wherein;
      Alk₂ through Alk₁₃ are independently C₁₋₈ straight or branched alkylene or alkenylene;
      q is an integer ranging from 1 to 8;
      Q is H, OH, NH₂, (CH2)ₜ OH, or R₁₃ₐCOOH, wherein t is an integer ranging from 1 to 8;
      R₈ through R₁₃ are independently H or C₁₋₈ straight or branched alkyl;
      R₁₃ₐ is C₁₋₈ straight or branched alkylene;
      R₁₄ is H, CH₃, or (CH₂)ₚ₁CH₃;
      R₁₅ is H, CH₃, (CH₂)ₚ₂CH₃ or (CH₂)ₘOH,
         wherein; p₁ and p₂ are independently integers from 1 to 7, and m is an integer from 1 to 8;
R₃ is Alk₁₄ArR₁₆,
   wherein;
      Alk₁₄ is C₁₋₈ straight or branched alkylene or alkenylene;
      Ar is a 5- or 6-member aromatic ring containing 0 to 4 heteroatoms selected from N, O, and S, or is a bicyclic 9- or 11-member aromatic ring containing 0 to 6 heteroatoms selected from N, O, and S;
      R₁₆ is H, OH, OR_{13b}, NO₂, NH₂, CN, Alk₁₅OH, Alk₁₆NH₂, NR₁₇R₁₈, NR₁₉COR₁₉ₐ, Alk₁₇COOR_{19b}, SO₂R_{19c}, SO₃H, PO₃H₂ or halogen;
         wherein;
         Alk₁₅ through Alk₁₇ are independently C₁₋₈ straight or branched alkylene or alkenylene;
         R_{13b} is H, or C₁₋₈ straight or branched alkyl;
         R₁₇, through R₁₉ and R₁₉ₐ through R_{19c} are independently H, an aromatic group, or C₁₋₈ straight or branched alkyl;
R₄ is

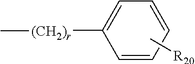

wherein;
   r is an integer from 1 to 20;
   R₂₀ is SO₃H, PO₃H₂, halogen, OR_{13c}, COOR_{13d}, NO₂, NR₂₁R₂₂,
   NR₂₃COR₂₃ₐ, Alk₁₈COOR_{19d}, SO₂R₁₉ₑ or Alk₁₈NR₂₄R₂₅ and when R₃ is other than (CH₂)_{q}(C₆H₄)Q, R₂₀ is as defined above or is H, OH, NH₂ Alk₁₉OH, Alk₂₀NH₂, or Alk₂₁COOH;
      wherein;
      Alk₁₉ through Alk₂₁ are independently C₁₋₈ straight or branched alkylene or alkenylene;
      R_{13c} and R_{13d} are independently C₁₋₈ straight or branched alkyl;
      R_{19d} and R₁₉ₑ are independently H, an aromatic group or C₁₋₈ straight or branched alkyl;
      R₂₁, through R₂₅ and R₂₃ₐ are independently H, an aromatic group or C₁₋₈ straight or branched alkyl;
         with one or more radioactive or non-radioactive label moieties attached thereto.

A second aspect is the use of a compound of formula (I) as an imaging agent in diagnostic procedures such as MRI and PET.

A third aspect is the use of a compound of formula (I) in a cell or receptor based assay.

### DETAILED DESCRIPTION OF EMBODIMENTS OF THE INVENTION

The present invention will now be described more fully hereinafter, in which embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

While the present invention is intended primarily for the treatment of human subjects, it will be appreciated that other subjects, particularly mammalian subjects such as dogs, cats, horses, rabbits, etc., can also be treated by the methods of the present invention for veterinary purposes.

“Halogen” as used herein refers to any suitable halo group, such as fluorine, chlorine, bromine, and iodine. Compounds as described above may be prepared in accordance with the techniques known in the art such as described in U.S. Pat. No. 5,719,279, U.S. Pat. No. 5,786,360, U.S. Pat. No. 5,739,331, U.S. Pat. No. 6,489,332, the techniques described in the Examples below, and variations of the foregoing that will be understandable to those skilled in the art of synthetic organic chemistry in light of the disclosure herein.

Specific examples of compounds of the present invention that can be prepared by such techniques include, but are not limited to, the following:
3-[2-[4-(5-aminopentyl-3-amidocarboxypropanoyl)]aminophenyl]ethyl]-8-benzyl-7-[2-ethyl(2-hydroxyethyl)amino]ethyl-1-propylxanthine,
3-[2-[4-(5-aminopentyl-3-amidocarboxypropanoyl)]aminophenyl]ethyl]-8-benzyl-7-(2,2-diethylamino]ethyl-1-propylxanthine,
3-[2-[4-(5-aminopentyl-3-amidocarboxypropanoyl)]aminophenyl]ethyl]-8-benzyl-7-(2,2-diethylamino]ethyl-1-propylxanthine, d-biotin amido adduct,
3-[2-[4-(5-aminopentyl-3-amidocarboxypropanoyl)]aminophenyl]ethyl]-8-benzyl-7-(2,2-diethylamino]ethyl-1-propylxanthine, Cy3B amido adduct,
3-[2-[4-(5-aminopentanoyl)aminophenyl]ethyl]-8-benzyl-7-[2-ethyl(2-hydroxyethyl)amino]ethyl-1-propylxanthine,
3-[2-[4-(5-aminopentanoyl)aminophenyl]ethyl]-8-benzyl-7-(2,2-diethylamino)ethyl-1-propylxanthine,
3-[2-[4-(5-aminopentanoyl)aminophenyl]ethyl]-8-benzyl-7-(2,2-diethylamino)ethyl-1-propylxanthine, d-biotin amido adduct,
3-[2-[4-(5-aminopentanoyl)aminophenyl]ethyl]-8-benzyl-7-(2,2-diethylamino)ethyl-1-propylxanthine, Cy3B amido adduct,
3-[4-(4-aminophenyl)butyl]-8-benzyl-7-(2-ethylamino)ethyl-1-pentylxanthine,
3-[4-(2-aminophenyl)butyl]-8-benzyl-7-(2-ethylamino)ethyl-1-propylxanthine,
3-[4-(3-aminophenyl)butyl]-8-benzyl-7-(2-ethylamino)ethyl-1-propylxanthine,
3-[4-(4-aminophenyl)butyl]-8-benzyl-7-[2-ethyl(2-hydroxyethyl)amino]ethyl-1-pentylxanthine,
3-[4-(2-aminophenyl)butyl]-8-benzyl-7-[2-ethyl(2-hydroxyethyl)amino]ethyl-1-propylxanthine,
3-[4-(3-aminophenyl)butyl]-8-benzyl-7-[2-ethyl(2-hydroxyethyl)amino]ethyl-1-propylxanthine,
3-[4-(3-aminophenyl)butyl]-8-benzyl-7-[2-ethyl(2-hydroxyethyl)amino]ethyl-1-(3-fluoro)propylxanthine,
3-[4-(3-aminophenyl)butyl]-8-benzyl-7-[2-ethyl(2-hydroxyethyl)amino]ethyl-1-(3,3,3-trifluoro)propylxanthine,
3-[4-(3-aminophenyl)butyl]-8-benzyl-7-[2-ethyl(2-hydroxyethyl)amino]ethyl-1-(1,1,2,2,3,3,3-heptafluoro)propylxanthine,
3-[2-(3-acetaminophenyl)ethyl]-7-[2-ethyl(2-hydroxyethyl)amino]ethyl-1-propyl-8-[(pyrimidin-5-yl)methyl]xanthine,
3-[2-(4-acetaminophenyl)ethyl]-7-[2-ethyl(2-hydroxyethyl)amino]ethyl-8-(3-methylsulfonobenzyl)-1-propylxanthine,
8-(3-aminobenzyl)-7-[2-ethyl(2-hydroxyethyl)amino]ethyl-1-pentyl-3-(2-phenylethyl)xanthine,
8-(3-aminobenzyl)-7-[2-ethyl(2-hydroxyethyl)amino]ethyl-3-(2-phenylethyl)-1-propylxanthine,
8-(2-aminobenzyl)-3-[2-(2-aminophenyl)ethyl]-7-[2-ethyl(2-hydroxyethyl)amino]ethyl-1-propylxanthine,
8-(2-aminobenzyl)-3-[2-(3-aminophenyl)ethyl]-7-[2-ethyl(2-hydroxyethyl)amino]ethyl-1-propylxanthine,
8-(2-aminobenzyl)-3-[2-(3-aminophenyl)ethyl]-7-[2-ethyl(2-hydroxyethyl)amino]ethyl-1-(3-fluoro)propylxanthine,
3-[2-[2-(6-aminohexanoyl)aminophenyl]ethyl]-8-benzyl-7-[2-ethyl(2-hydroxyethyl)amino]ethyl-1-(3-methoxypropyl)xanthine,
3-[2-(4-aminophenyl)ethyl]-8-benzyl-1-butyl-7-[2-ethyl(2-hydroxyethyl)amino]ethylxanthine,
3-[2-(2-aminophenyl)ethyl]-8-benzyl-7-[2-ethyl(2-hydroxyethyl)amino]ethyl-1-pentylxanthine,
3-[2-(4-aminophenyl)ethyl]-8-benzyl-7-[2-ethyl(2-hydroxyethyl)amino]ethyl-1-propylxanthine,
3-[2-(4-aminophenyl)ethyl]-8-benzyl-7-[2-ethyl(2-hydroxyethyl)-amino]ethyl-1-pentylxanthine,
3-[2-(4-aminophenyl)ethyl]-8-benzyl-7-(2-ethylamino)ethyl-1-pentylxanthine,
3-[2-(4-aminophenyl)ethyl]-8-benzyl-7-(2,2-diethylamino)ethyl-1-propylxanthine,
3-[2-(4-aminophenyl)ethyl]-8-benzyl-7-[2-methyl(2-hydroxyethyl)amino]ethyl-1-propylxanthine,
3-[2-(4-aminophenyl)ethyl]-8-benzyl-7-(2-methylamino)ethyl-1-propylxanthine,
3-[2-(4-aminophenyl)ethyl]-8-benzyl-7-(2,2-dimethylamino)ethyl-1-propylxanthine,
3-[2-(4-aminophenyl)ethyl]-8-benzyl-7-[2-ethyl(2-hydroxyethyl)amino]ethyl-1-(3-fluoro)propylxanthine,
3-[2-(4-aminophenyl)ethyl]-8-(3-chlorobenzyl)-7-[2-ethyl(2-hydroxyethyl)amino]ethyl-1-propylxanthine,
3-[2-(2-aminophenyl)ethyl]-8-benzyl-1-(3-dimethylaminopropyl)-7-[2-ethyl(2-hydroxyethyl)amino]ethylxanthine,
3-[2-(2-aminophenyl)ethyl]-8-benzyl-1-(3-dimethylaminopropyl)-7-(2,2-diethylamino)ethylxanthine,
3-[2-(2-aminophenyl)ethyl]-8-benzyl-1-(3-dimethylaminopropyl)-7-(2-ethylamino)ethylxanthine,
3-[2-(2-aminophenyl)ethyl]-8-benzyl-7-[2-ethyl(2-hydroxyethyl)amino]ethyl-1-(3-methoxypropyl)xanthine,
3-[2-(3-aminophenyl)ethyl]-8-benzyl-7-[2-ethyl(2-hydroxyethyl)amino]ethyl-1-(3-methoxypropyl)xanthine,
3-[2-(3-aminophenyl)ethyl]-8-benzyl-7-[2-ethyl(2-hydroxyethyl)amino]ethyl-1-(3-methylsulfonopropyl)xanthine,
3-[2-(3-aminophenyl)ethyl]-1-butyl-7-[2-ethyl(2-hydroxyethyl)amino]ethyl-8-[(pyridazin-4-yl)methyl]xanthine,
3-[2-(4-amino-3-chlorophenyl)ethyl]-7-[2-ethyl(2-hydroxyethyl)amino]ethyl-1-propyl-8-[(pyridazin-4-yl)methyl]xanthine,
3-[2-(4-amino-2-chlorophenyl)ethyl]-7-[2-ethyl(2-hydroxyethyl)amino]ethyl-1-propyl-8-[(pyridazin-4-yl)methyl]xanthine,
3-[2-(4-amino-2-fluorophenyl)ethyl]-7-[2-ethyl(2-hydroxyethyl)amino]ethyl-1-propyl-8-[(1H-pyrrol-3-yl)methyl]xanthine,
3-[2-(3-aminophenyl)ethyl]-7-[2-ethyl(2-hydroxyethyl)amino]ethyl-1-propyl-8-[(1H-1,3,4-triazol-5-yl)methyl]xanthine,
3-[2-(2-aminophenyl)ethyl]-7-[2-ethyl(2-hydroxyethyl)amino]ethyl-1-propyl-8-[(1H-1,2,4-triazol-5-yl)methyl]xanthine,
3-[2-(3-aminophenyl)ethyl]-7-[2-ethyl(2-hydroxyethyl)amino]ethyl-8-[(1,2,4-oxadiazol-5-yl)methyl]-1-propylxanthine,
3-[2-(4-aminophenyl)ethyl]-7-[2-ethyl(2-hydroxyethyl)amino]ethyl-1-propyl-8-[(oxazol-2-yl)methyl]xanthine,
3-[2-(2-aminophenyl)ethyl]-7-[2-ethyl (2-hydroxyethyl)amino]ethyl-8-[(isoxazol-4-yl)methyl]-1-propylxanthine,
3-[2-(2-aminophenyl)ethyl]-8-[(5-chloroisoxazol-4-yl)methyl]-7-[2-ethyl(2-hydroxyethyl)amino]ethyl-1-propylxanthine,
3-[2-(4-aminophenyl)ethyl]-8-(2,4-difluorobenzyl)-7-[2-ethyl (2-hydroxyethyl)amino]ethyl-1-propylxanthine,
3-[2-(2-aminophenyl)ethyl]-7-[2-ethyl (2-hydroxyethyl)amino]ethyl-8-[(5-fluoroisoxazol-4-yl)methyl]-1-pentylxanthine,
3-[2-(4-aminophenyl)ethyl]-7-[2-ethyl(2-hydroxyethyl)amino]ethyl-8-[(4-fluoro-2-oxazolyl)methyl]-1-propylxanthine,
3-[2-(4-aminophenyl)ethyl]-7-[2-ethyl(2-hydroxyethyl)amino]ethyl-8-[(isothiazol-3-yl)methyl]-1-propyl-xanthine,
3-[2-(3-aminophenyl)ethyl]-7-[2-ethyl(2-hydroxyethyl)amino]ethyl-1-propyl-8-[(pyrimidin-2-yl)methyl]xanthine,
3-[2-(2-aminophenyl)ethyl]-7-[2-ethyl(2-hydroxyethyl)amino]ethyl-8-[(4-fluoro-3-isothiazolyl)methyl]-1-propylxanthine,
3-[2-(4-aminophenyl)ethyl]-7-[2-ethyl (2-hydroxyethyl)amino]ethyl-8-[(5-fluoropyrimidin-2-yl)methyl]-1-propylxanthine,
3-[2-(3-aminophenyl)ethyl]-7-[2-ethyl(2-hydroxyethyl)amino]ethyl-8-[(1,3,4-oxadiazol-5-yl)methyl]-1-pentylxanthine,
3-[2-(4-aminophenyl)ethyl]-7-[2-ethyl(2-hydroxyethyl)amino]ethyl-1-propyl-8-[(1H-pyrazol-3-yl)methyl]xanthine,
3-[2-(3-aminophenyl)ethyl]-7-[2-ethyl(2-hydroxyethyl)amino]ethyl-1-propyl-8-[(1H-pyrazol-3-yl)methyl]xanthine,
3-[2-(4-aminophenyl)ethyl]-7-[2-ethyl(2-hydroxyethyl)amino]ethyl-1-pentyl-8-[(1H-pyrazol-3-yl)methyl]xanthine,
3-[2-(2-aminophenyl)ethyl]-7-[2-ethyl(2-hydroxyethyl)amino]ethyl-1-propyl-8-[(pyrazin-2-yl)methyl]xanthine,
3-[2-(2-aminophenyl)ethyl]-1-butyl-7-[2-ethyl(2-hydroxyethyl)amino]ethyl-8-[(3-fluoropyrazin-2-yl)methyl]xanthine,
3-[2-(2-aminophenyl)ethyl]-7-[2-ethyl(2-hydroxyethyl)amino]ethyl-8-[(3-fluoropyrazin-2-yl)methyl]-1-pentylxanthine,
3-[2-(2-aminophenyl)ethyl]-7-[2-ethyl(2-hydroxyethyl)amino]ethyl-8-[(3-fluoropyrazin-2-yl)methyl]-1-propylxanthine,
3-[2-(4-aminophenyl)ethyl]-7-[2-ethyl(2-hydroxyethyl)amino]ethyl-1-pentyl-8-[(2-fluoro-1H-pyrazol-3-yl)methyl]xanthine,
3-[2-(4-aminophenyl)ethyl]-7-[2-ethyl(2-hydroxyethyl)amino]ethyl-1-propyl-8-[(1H-pyrrol-3-yl)methyl]xanthine,
3-[2-(2-aminophenyl)ethyl]-7-[2-ethyl(2-hydroxyethyl)amino]ethyl-1-propyl-8-[(1H-tetrazol-5-yl)methyl]xanthine,
3-[2-(3-aminophenyl)ethyl]-7-[2-ethyl(2-hydroxyethyl)amino]ethyl-1-propyl-8-[(furan-3-yl)methyl]xanthine,
3-[2-(4-aminophenyl)ethyl]-7-[2-ethyl(2-hydroxyethyl)amino]ethyl-1-propyl-8-[(furan-2-yl)methyl]xanthine,
3-[2-(4-aminophenyl)ethyl]-7-[2-ethyl(2-hydroxyethyl)amino]ethyl-1-propyl-8-[(thiophen-3-yl)methyl]xanthine,
3-[6-(4-aminophenyl)hexyl]-8-benzyl-7-(2-ethylamino)ethyl-1-pentylxanthine,
3-[6-(2-aminophenyl)hexyl]-8-benzyl-7-(2-ethylamino)ethyl-1-propylxanthine,
3-[6-(3-aminophenyl)hexyl]-8-benzyl-7-(2-ethylamino)ethyl-1-propylxanthine,
3-[6-(3-aminophenyl)hexyl]-8-benzyl-7-(2-ethylamino)ethyl-1-(3-fluoro)propylxanthine,
3-[6-(4-aminophenyl)hexyl]-8-benzyl-7-[2-ethyl(2-hydroxyethyl)amino]ethyl-1-pentylxanthine,
3-[6-(2-aminophenyl)hexyl]-8-benzyl-7-[2-ethyl(2-hydroxyethyl)amino]ethyl-1-propylxanthine,
3-[6-(3-aminophenyl)hexyl]-8-benzyl-7-[2-ethyl(2-hydroxyethyl)amino]ethyl-1-propylxanthine,
3-[6-(3-aminophenyl)hexyl]-8-benzyl-7-[2-ethyl(2-hydroxyethyl)amino]ethyl-1-(3-fluoro)propylxanthine,
8-benzyl-3-[2-(3-aminophenyl)ethyl]-7-[2-ethyl(2-hydroxyethyl)amino]ethyl-1-propylxanthine,
8-benzyl-3-[2-(3-chlorophenyl)ethyl]-7-[2-ethyl(2-hydroxyethyl)amino]ethyl-1-propylxanthine,
8-benzyl-3-[2-(2,4-difluorophenyl)ethyl]-7-[2-ethyl(2-hydroxyethyl)amino]ethyl-1-pentylxanthine
8-benzyl-7-[2-ethyl(2-hydroxyethyl)amino]ethyl-3-[2-(3-nitrophenyl)ethyl]-1-propylxanthine,
8-benzyl-7-(2-ethylamino)ethyl-3-[2-(3-nitrophenyl)ethyl]-1-propylxanthine,
8-benzyl-7-(2,2-diethylamino)ethyl-3-[2-(3-nitrophenyl)ethyl]-1-propylxanthine,
8-benzyl-7-[2-ethyl(2-hydroxyethyl)amino]ethyl-3-[2-(isothiazol-3-yl)ethyl]-1-propylxanthine,
8-benzyl-7-[2-ethyl(2-hydroxyethyl)amino]ethyl-3-[2-(thiazol-3-yl)ethyl]-1-propylxanthine,
8-benzyl-7-[2-ethyl(2-hydroxyethyl)amino]ethyl-3-[2-(isoxazol-3-yl)ethyl]-1-propylxanthine,
8-benzyl-7-[2-ethyl(2-hydroxyethyl)amino]ethyl-3-[2-(1,3,4-oxadiazol-5-yl)ethyl]-1-pentylxanthine,
8-benzyl-7-[2-ethyl(2-hydroxyethyl)amino]ethyl-3-[2-(1,2,4-oxadiazol-5-yl)ethyl]-1-propylxanthine,
8-benzyl-7-[2-ethyl(2-hydroxyethyl)amino]ethyl-3-[2-(4-fluorophenyl)ethyl]-1-pentylxanthine,
8-benzyl-7-[2-ethyl(2-hydroxyethyl)amino]ethyl-3-[2-(4-nitrophenyl)ethyl]-1-propylxanthine,
8-benzyl-7-[2-ethyl(2-hydroxyethyl)amino]ethyl-3-[2-phenylethyl]-1-pentylxanthine,
8-benzyl-7-[2-ethyl(2-hydroxyethyl)amino]ethyl-3-[2-phenylethyl]-1-propylxanthine,
3-[2-(4-bromophenyl)ethyl]-7-[2-ethyl(2-hydroxyethyl)amino]ethyl-1-propyl-8-[(4-pyridyl)methyl]xanthine,
3-[2-(4-chlorophenyl)ethyl]-7-[2-ethyl(2-hydroxyethyl)amino]ethyl-1-propyl-8-[(4-pyridyl)methyl]xanthine,
3-[2-(2,4-diaminophenyl)ethyl]-7-[2-ethyl(2-hydroxyethyl)amino]ethyl-8-[(5-fluoro-2-oxazolyl)methyl]-1-propylxanthine,
7-(2,2-diethylamino)ethyl-3-(2-phenylethyl)-1-propyl-8-[(2-pyridyl)methyl]xanthine,
7-(2,2-diethylamino)ethyl-3-[2-(3-fluorophenyl)ethyl]-8-[(1,3,4-oxadiazol-5-yl)methyl]-1-propylxanthine,
7-(2,2-diethylamino)ethyl-3-[2-(3-nitrophenyl)ethyl]-1-propyl-8-[(pyridazin-4-yl)methyl]xanthine,
7-(2,2-diethylamino)ethyl-3-(2-phenylethyl)-1-propyl-8-[(1H-pyrazol-3-yl)benzyl]xanthine,
7-(2-ethylamino)ethyl-3-(2-phenylethyl)-1-propyl-8-[(2-pyridyl)methyl]xanthine,
7-(2-ethylamino)ethyl-3-[2-(3-nitrophenyl)ethyl]-8-[(1,3,4-oxadiazol-5-yl)methyl]-1-propyllxanthine,
7-(2-ethylamino)ethyl-3-[2-(2-nitrophenyl)ethyl]-8-[(4-fluoro-3-isothiazolyl)methyl]-1-propylxanthine,
7-(2-ethylamino)ethyl-3-[2-(2-fluorophenyl)ethyl]-1-propyl-8-[(pyrazin-2-yl)methyl]xanthine,
7-[2-ethyl(2-hydroxyethyl)amino]ethyl-3-[2-(2-fluorophenyl)ethyl]-1-propyl-8-[(pyrazin-2-yl)methyl]xanthine,
7-[2-ethyl(2-hydroxyethyl)amino]ethyl-3-[2-(3-fluorophenyl)ethyl]-8-[(1,3,4-oxadiazol-5-yl)methyl]-1-propylxanthine,
7-[2-ethyl(2-hydroxyethyl)amino]ethyl-3-[2-(4-nitrophenyl)ethyl]-1-propyl-8-[(1H-pyrazol-3-yl)methyl]xanthine,
7-[2-ethyl(2-hydroxyethyl)amino]ethyl-3-[2-(3-nitrophenyl)ethyl]-8-[(1,3,4-oxadiazol-5-yl)methyl]-1-propyllxanthine,
7-[2-ethyl(2-hydroxyethyl)amino]ethyl-3-[2-(2-nitrophenyl)ethyl]-1-propyl-8-[(1H-1,2,4-triazol-5-yl)methyl]xanthine,
7-[2-ethyl(2-hydroxyethyl)amino]ethyl-8-[(4-fluoro-3-isothiazolyl)methyl]-3-[2-(2-nitrophenyl)ethyl]-1-propylxanthine,
7-[2-ethyl(2-hydroxyethyl)amino]ethyl-3-[2-(3-nitrophenyl)ethyl]-1-propyl-8-[(pyridazin-4-yl)methyl]xanthine,
7-[2-ethyl(2-hydroxyethyl)amino]ethyl-3-[2-(3-nitrophenyl)ethyl]-8-[(1,2,4-oxadiazol-5-yl)methyl]-1-propylxanthine,
7-[2-ethyl(2-hydroxyethyl)amino]ethyl-8-[(1,2,4-oxadiazol-3-yl)benzyl]-3-(2-phenylethyl)-1-propylxanthine,
7-[2-ethyl(2-hydroxyethyl)amino]ethyl-8-[(1,3,4-oxadiazol-5-yl)benzyl]-3-(2-phenylethyl)-1-propylxanthine,
7-[2-ethyl(2-hydroxyethyl)amino]ethyl-3-(2-phenylethyl)-1-propyl-8-[(1H-pyrazol-3-yl)benzyl]xanthine,
7-[2-ethyl(2-hydroxyethyl)amino]ethyl-1-pentyl-3-(2-phenylethyl)-8-[(3-pyridyl)methyl]xanthine,
7-[2-ethyl(2-hydroxyethyl)amino]ethyl-3-(2-phenylethyl)-1-propyl-8-[(2-pyridyl)methyl]xanthine, and
7-[2-ethyl(2-hydroxyethyl)amino]ethyl-3-(2-phenylethyl)-1-propyl-8-[(4-pyridyl)methyl]xanthine
3-[2-(4-aminophenyl)ethyl]-8-benzyl-7-(2-ethylamino)ethyl-1-propylxanthine (example 5),
3-[2-(4-aminophenyl)ethyl]-8-benzyl-7-(2,2-diethylamino)ethyl-1-propylxanthine (example 6),
3-[2-(4-aminophenyl)ethyl]-7-[2-ethyl(2-hydroxyethyl)amino]ethyl-8-(4-fluorobenzyl)-1-propylxanthine (example 7),
3-[2-(4-aminophenyl)ethyl]-7-[2-ethyl(2-hydroxyethyl)amino]ethyl-1-propyl-8-[(3-pyridyl)methyl]xanthine (example 8),
3-[2-(4-aminophenyl)ethyl]-7-(2-ethylamino)ethyl-1-propyl-8-[(3-pyridyl)methyl]xanthine (example 9),
3-[2-(4-aminophenyl)ethyl]-7-(2,2-diethylamino)ethyl-1-propyl-8-[(3-pyridyl)methyl]xanthine (example 10),
3-[2-(4-aminophenyl)ethyl]-7-[2-ethyl(2-hydroxyethyl)amino]ethyl-1-propyl-8-[(thiophen-2-yl)methyl]xanthine (example 11),
3-[2-(4-aminophenyl)ethyl]-7-[2-ethyl(2-hydroxyethyl)amino]ethyl-1-propyl-8-[(4-thiazolyl)methyl]xanthine (example 12),
3-[2-(4-aminophenyl)ethyl]-7-[2-ethyl(2-hydroxyethyl)amino]ethyl-1-propyl-8-[(1H-tetrazol-5-yl)methyl]xanthine (example 13),
3-[2-(4-aminophenyl)ethyl]-7-(2-ethylamino)ethyl-1-propyl-8-[(1H-tetrazol-5-yl)methyl]xanthine (example 14),
3-[2-(4-aminophenyl)ethyl]-7-(2,2-diethylamino)ethyl-1-propyl-8-[(1H-tetrazol-5-yl)methyl]xanthine (example 15),
3-[2-(4-aminophenyl)ethyl]-7-[2-ethyl(2-hydroxyethyl)amino]ethyl-8-(4-methylsulfonobenzyl)-1-propylxanthine (example 16),
3-[2-(4-aminophenyl)ethyl]-7-(2-ethylamino)ethyl-8-(4-methylsulfonobenzyl)-1-propylxanthine (example 17),
3-[2-(4-aminophenyl)ethyl]-7-(2,2-diethylamino)ethyl-8-(4-methylsulfonobenzyl)-1-propylxanthine (example 18),
3-[2-(4-aminophenyl)ethyl]-8-benzyl-7-[2-ethyl(2-hydroxyethyl)amino]ethyl-1-(3-methoxypropyl)xanthine (example 19),
3-[2-(4-aminophenyl)ethyl]-8-benzyl-7-(2-ethylamino)ethyl-1-(3-methoxypropyl)xanthine (example 20),
3-[2-(4-aminophenyl)ethyl]-8-benzyl-7-(2,2-diethylamino)ethyl-1-(3-methoxypropyl)xanthine (example 21),
3-[2-(4-aminophenyl)ethyl]-8-benzyl-1-(3-dimethylaminopropyl)-7-[2-ethyl(2-hydroxyethyl)amino]ethylxanthine (example 22),
3-[2-(4-aminophenyl)ethyl]-8-benzyl-1-(3-dimethylaminopropyl)-7-(2-ethylamino)ethylxanthine (example 23),
3-[2-(4-aminophenyl)ethyl]-8-benzyl-7-(2,2-diethylamino)ethyl-1-(3-dimethylaminopropyl)xanthine (example 24),
3-[2-[4-(6-aminohexanoyl)aminophenyl]ethyl]-8-benzyl-7-(2,2-diethylamino)ethyl-1-propylxanthine (example 25),
3-[2-[4-(6-aminohexanoyl)aminophenyl]ethyl]-8-benzyl-7-(2,2-diethylamino)ethyl-1-propylxanthine, Cy3B amido adduct (example 26),
3-[2-[4-(6-aminohexyl-3-amidocarboxypropanoyl)]aminophenyl]ethyl]-8-benzyl-7-(2,2-diethylamino]ethyl-1-propylxanthine (example 27),
3-[2-[4-(6-aminohexyl-3-amidocarboxypropanoyl)]aminophenyl]ethyl]-8-benzyl-7-(2,2-diethylamino]ethyl-1-propylxanthine, Cy3B amido adduct (example 28),
3-[2-[4-(6-aminohexyl-3-amidocarboxypropanoyl)]aminophenyl]ethyl]-8-benzyl-7-(2,2-diethylamino]ethyl-1-propylxanthine, d-biotin amido adduct (example 29),
3-[2-(4-aminophenyl)ethyl]-8-benzyl-7-[1³H-(2³H-[2-ethyl(2-hydroxyethyl)amino]ethyl]-1-propylxanthine (example 30),
3-[2-(4-aminophenyl)ethyl]-8-benzyl-7-[1³H-[2³H-(2-ethylamino)ethyl]-1-propylxanthine (example 31),
3-[4-(4-aminophenyl)butyl]-8-benzyl-7-[2-ethyl(2-hydroxyethyl)amino]ethyl-1-propylxanthine (example 32),
3-[4-(4-aminophenyl)butyl]-7-[2-ethyl(2-hydroxyethyl)amino]ethyl-1-propyl-8-(4-sulfonoxybenzyl)xanthine (example 33),
3-[4-(4-aminophenyl)butyl]-7-(2-ethylamino)ethyl-1-propyl-8-(4-sulfonoxybenzyl)xanthine (example 34),
3-[4-(4-aminophenyl)butyl]-7-(2,2-diethylamino)ethyl-1-propyl-8-(4-sulfonoxybenzyl)xanthine (example 35),
3-[4-(4-aminophenyl)butyl]-7-(2,2-dimethylamino)ethyl-1-propyl-8-(4-sulfonoxybenzyl)xanthine (example 36),
3-[2-(4-aminophenyl)ethyl]-8-benzyl-7-[2-ethyl(2-hydroxyethyl)amino]ethyl-1-(3-methoxyethyl)xanthine,
3-[2-(4-aminophenyl)ethyl]-8-benzyl-7-(2-ethylamino)ethyl-1-(3-methoxyethyl)xanthine,
3-[2-(4-aminophenyl)ethyl]-8-benzyl-7-(2,2-diethylamino)ethyl-1-(3-methoxyethyl)xanthine,
3-[2-(4-aminophenyl)ethyl]-8-benzyl-1-(3-dimethylaminoethyl)-7-[2-ethyl(2-hydroxyethyl)amino]ethylxanthine,
3-[2-(4-aminophenyl)ethyl]-8-benzyl-1-(3-dimethylaminoethyl)-7-(2-ethylamino)ethylxanthine,
3-[2-(4-aminophenyl)ethyl]-8-benzyl-7-(2,2-diethylamino)ethyl-1-(3-dimethylaminoethyl)xanthine, or
3-[4-(4-aminophenyl)butyl]-7-[2-methyl(2-hydroxyethyl)amino]ethyl-1-propyl-8-(4-sulfonoxybenzyl)xanthine.

The compounds of formula (I) may form salts with both organic and inorganic acid and bases. Likewise, the compounds of formula (I) may form solvates including hydrates. All salts and solvates of the compounds of formula (I) are within the scope of the present invention. While pharmaceutically acceptable salts and solvates are useful for the treatment of mammals, including humans, non-pharmaceutically salts and solvates may be useful as chemical intermediates, and thus, are within the scope of the present invention. Examples of suitable acids for pharmaceutically acceptable salt formation include, but are not limited to, hydrochloric, sulfuric, phosphoric, acetic, citric, oxalic, malonic, salicylic, ascorbic, maleic, methanesulfonic, benzenesulfonic, p-toluenesulfonic and the like. Any of the amine acid addition salts may also be used. The salts are prepared by contacting the free base form of the compound with an appropriate amount of the desired acid in a manner known to one skilled in the art.

Examples of suitable bases for pharmaceutically acceptable salt formation include, but are not limited to, ammonium hydroxide, sodium hydroxide, sodium carbonate, sodium bicarbonate, potassium hydroxide, calcium hydroxide, ammonia, organic amines such as triethylamine, and the like. The salts may be prepared by contacting the free acid form of the compound with an appropriate amount of the desired base in a manner known to one skilled in the art. An example of a suitable solvate is a hydrate. Solvates may be prepared by any appropriate method of the art.

The compounds of formula (I) may be administered per se or in the form of acid or basic salts, hydrates, solvates and pro-drugs thereof, in accordance with known techniques, to carry out the methods described herein.

Active compounds of the invention may be provided in the form of prodrugs. The term “prodrug” refers to compounds that are transformed in vivo to yield the parent compound of the above formulae, for example, by hydrolysis in blood. A thorough discussion is provided in T. Higuchi and V. Stella, Prodrugs as Novel Delivery Systems, Vol. 14 of the A.C.S. Symposium Series and in Edward B. Roche, ed., Bioreversible Carriers in Drug Design, American Pharmaceutical Association and Pergamon Press, 1987. See also U.S. Pat. No. 6,680,299. Examples include, but are not limited to, a prodrug that is metabolized in vivo by a subject to an active drug having at least some of the activity of the active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

The compounds of the present invention are useful as diagnostic probes, including probes for diagnostic assays and imaging. Accordingly, the invention also provides A₁ adenosine receptor antagonist compounds with radioactive or non-radioactive labels suitable for executing such assays and imaging measurements. Labeled compounds are useful as diagnostic probes or conjugates, and to obtain quantitative measurements of the A₁ adenosine receptor antagonist compounds. As used herein, the term “diagnostic probes” refers to those materials which are useful for enhancing the quantitative analysis of the A₁ adenosine receptor compounds of the invention and to enhance the quantitative analysis of A₁ adenosine receptors.

Examples of such assay-type probes and their diagnostic uses are described in Jacobson, et al., U.S. Pat. No. 5,248,770 (770).and in Baker, et al.,(2010), Br J Pharmacol, 159, 772-786. Such probes are useful because they have little adverse effect on the affinity of the compounds of the present invention for the A₁ adenosine receptor. Nuclear markers (also referred to a “labels”) include, but are not limited to, nuclear spin markers, e.g. a ¹⁹F magnetic resonance imaging (MRI) probe, radioactive markers, e.g., ¹⁸F, ¹¹C, ¹⁵N, ¹²⁵I, and ³H (also referred to as “tritium”) isotope marker, and complexes of metal atoms or metal ions and chelating agents. Chelating agents and chelates are well known in the art, see for example, the definition of “chelate in Hawley's Condensed Chemical Dictionary, 13^{th} edition, 240, (1997). Typically the metal atom or metal ion in the complex will have a heavy, radioactive nucleus which serves as a marker atom. Additionally, atoms in the lanthanide series may serve as marker atoms. The marker atoms may be chemically bonded to, conjugated, or complexed, e.g. chelated, with, a compound of formula (I) or may be one of the integral carbon or heteroatoms of a compound of formula (I). Additionally, these marker atoms may be linked to a compound of formula (1) by spacer or chelate moieties.

Such labeled compounds can be used for in vitro or in vivo imaging of A₁ adenosine receptors, especially in tissues, including but not limited to the brain, heart, liver, kidney, and lungs to obtain quantitative measurements of A₁ adenosine receptors and determine the distribution and regional binding characteristics of these receptors in tissue. These assay-type probes may be used, inter alia, in connection with such diagnostic techniques as MRI and positron emission tomography (PET), and single photon emission computed tomography (SPECT). See, for example, Myer, et al.(2004)J Cerebral Blood Flow & Metabolism 24:323-333, Wakabayashi, et al.(2000), Nuclear Med & Biol 27:401-406, Ishiwata, et al., (2010) Ger Geront Int Suppl 1:S180-S196, and Bauer and Ishiwata, Adenosine receptors in health and disease, Hand Exp Pharmacol, 193, 617-642 (2009). An exemplary metal ion is a radioactive isotope of technetium or indium. An exemplary chelating agent is diethylenetriamine pentaacetic acid.

Various non-radioactive materials can be used in labeling the present A₁ adenosine receptor compounds. Typically, these materials exhibit properties that are easily observable when exposed to electromagnetic radiation. For example, such a labeling material may strongly fluoresce when exposed to ultraviolet light, phosphoresce after such exposure, or appear intensely colored in visible light. Thus, compounds, to which these materials are attached, e.g., a present A₁ adenosine receptor compound described by formula (I) may be traced by electromagnetic related properties of the labeling material. Luminescence generally refers to light emission from a chemical species induced chemically, electrically, or photonically, e.g. exposure to electromagnetic radiation, and is a general term which includes luminescence and phosphorescence.

Numerous examples of both radioactive and non-radioactive materials used in labeling A₁ adnenosine receptors are presented in U.S. Pat. No. 5,248,770. Biotin is a well known non-radioactive label for such probes, as described in R. W. Old et al. Principals of Gene Manipulation, 4th ed: 328-331 (1989). To facilitate labeling the compounds with biotin or any other appropriate label, a spacer, also known as a linker, component or moiety may be added (e.g. conjugated) to a compound of formula (I) of the present invention by any suitable method taught in the art, e.g. see U.S. Pat. No. 5,248,770 or Baker et al., supra. Exemplary spacer, or linker, moieties include, but are not limited to, an oligopeptide, triglycidyl, N-hydroxysuccinimide ester, succinimidyl-thiohexane (6-thiohexyl-3-amidocarboxypropanoyl), succinimidyl hexamethyleneamine (6-aminohexyl-3-amidocarboxypropanoyl), succinimidyl-cadaverine (5-aminopentyl-3-amidocarboxypropanoyl), and succinimidyl-hexylmaleimide (6-N-maleimidohexyl-3-amidocarboxypropanoyl).

A non-radioactive label, e.g., biotin, may be bonded to any suitable spacer linkage provided by substituents on the compound structure in accordance with any suitable technique taught in the art. For example, referring to the compounds of formula (I) as defined herein, biotin may be bonded to one or more of the hydroxy groups, amino groups or carboxyl groups present such as at the R₁ through R₄ positions on the compound. Additionally, the biotin may be bonded to one or more of the hydroxyl groups that may be present at the R₁ through R₄ positions on the compound. The biotin-labeled probes may be detected through appropriate and well known analytical techniques

Fluorescent compounds, typically fluorescent dyes, may also be employed as a non-radioactive labels and are applied to appropriate locations on the compounds of the invention as described above. Such dyes include, but are not limited to, tetramethylrhodamine, fluorescein isothiocyanate, Cy3, (see Waggoner, et al., U.S. Pat. No. 5,268,486, Dec. 7, 1993) or Cy3B (see Waggoner et al., U.S. Pat. No. 6,133,445, Oct. 17, 2000) and mixtures thereof. Other non-radioactive materials include for example, nitrobenzoxadiazole; 2,2,6,6-tetramethyl-piperindinyloxy-4-isothiocyanate; luminescent dyes; obelin; and mixtures thereof, which may be applied in an analogous manner as fluorescent compounds.

The skilled artisan will appreciate that also within the scope of the invention is the use of the compounds of formula (I) labeled with a radioactive or non-radioactive label in in vitro assays. For example, such labeled compounds may be used in clinical cell based assays and in receptor based assays, see Baker et al., supra and Patel, et al. (1988), Mol Pharmacol 33:585-591. Such assays include, but are not limited to, radioligand binding assays, high throughput screening assays, and flow cytometry based assays, for example fluorescence-activated cell sorting (FACS) based assays. Examples of such assays include, but are not limited to, radioimmunoassay and enzyme-linked immunosorbent assays (ELISA) (see, e.g., Nelson, et al., Lehninger Principles of Biochemistry, 231, (2000).

The introduction of time resolved fluorescence (TRF) technologies, including fluorophores with optimal photophysical properties, e.g. high energy transfer efficiency, and that can be tagged to biomolecules or to compounds of formula (I) of the invention, as well as improvements in instrumentation and gating techniques over conventional methods of fluorescent measurement, have provided the basis for a new generation of fluorescence-based assays, i.e. TRF based assays. “Time resolved fluorescence assays” involves the use of one or more long-lived fluorophores combined with time-resolved detection (a delay between excitation and emission detection) which allows for detection without major fluorescence interferences. The principle of use of TRF is described in U.S. Pat. Nos. 4,374,120 and 4,058,732 and Soini and Hemmila (1979), Clin Chem 25:353-361.

TRF assays are currently either heterogeneous or homogeneous in nature. In homogeneous TRF (HTRF) assays, two labeled partners with fluorophores are required for energy transfer. This energy transfer occurs only when the two molecules are in direct proximity to one another. The first fluorophore acts as an energy donor and second fluorophore acts as an energy acceptor. The efficiency of the energy transfer is a function of the distance between the long-lived fluorescence donor and the short-lived fluorescence acceptor dyes. The most commonly used donor lanthanides used in TRF resonance energy transfer (TR-FRET) assays are europium and terbium. Other donor lanthanides include samarium and dysprosium. There are a number of resonance energy acceptors including, XL665 (allophycocyanin), d2, phycobiliprotein, tetramethylrhodamine, fluorescein, thionine, R phycocyanin, phycoerythrocyanine, C phycoerythrin, and others. Further examples suitable for use in the present invention can be found in U.S. Pat. No. 6,908,769 incorporated herein by reference in its entirety. Commonly used energy transfer acceptors in HTRF assays are d2 or XL665. A typical donor/acceptor pair in an HTRF assay that produces high FRET efficiency at excitation of 337 nm is europium cryptate as the donor with an emission of 620 nm and XL665 as the acceptor with an emission of 665 nm. The natural short-lived fluorescent emission of the free acceptor, XL665, compared to the long-lived emission in the energy transfer process (due to the long-lived fluorescent lifetime of europium cryptate as the donor) allows a clear distinction between bound (which occurs during energy transfer when the molecule tagged with XL665 comes into close proximity to the molecule tagged with europium cryptate) and free XL665.

Intrinsic or extrinsic components of an assay may be conjugated covalently or non-covalently to labels for TRF and serve as diagnostic probes. Lanthanides have a number of properties that make them attractive for bioanalytical measurements and analysis, including ligand receptor interactions. See for example, Hemmila and Mukkala (2001), Crit. Rev Clin Lab Sci 38:441-519 and Handl and Gillies (2005), Life Sci 77:361-371. For example, paramagnetic Gd may be used as a contrast-enhancing agent in MRI; radioactive lanthanide isotopes may have tissue selectivity and used as radiotherapeutic agents, such as the polyphosphonate chelate of ¹⁵³Sm; and, photoluminescence, time-resolved electroluminescence detection of Tb is may be used for time-resolved electrogenerated labeling in immunoassays (Hemmila and Mukkala supra,

When chelated with a suitable light absorbing ligand, lathanides express luminescence with long decay times, narrow-banded emissions, and large Stoke's shifts. Important factors to consider when designing fluorescent lanthanide chelates include chelate formation, thermodynamic and kinetic stability, coupling chemistry, light harvesting (“antennae effect”), biocompatibility requirements of the biological system, and optimized emission profile. See for example, Hemmila and Mukkala supra, Handl and Gillies supra, Hemilla and Laitala (2005), J Fluores 15:529-542, and Moore et al., (2009) Acc Chem Res 42:542-552. An ideal chelate would provide suitable ligand triplet state to avoid quench reactions, protection against O—H and other vibrational deactivation processes, and prevent access of water to the inner sphere.

The type of chelating ligand used in lanthanide based assays depend on the type of application, chelate formation moiety, or by their antenna part (Hemilla and Laitala supra. Enhancement-based assays may be composed of polycarboxylate (EDTA and DTPA), diethylenetriaminetetraacetic acid (DTTA) based labeling reagents and β-diketone-based chelators as enhancement ligands. See for example, Handl and Gillies supra and Hemilla and Laitala supra. Commonly used antenna moieties are pyridine, bipyridine, terpyridine, salicylate, coumarin derivatives, phenanthroline, and various pyrazole deriviatives.

Fluorescent assays may use dissociative fluorescence, where nonfluorescent chelates are used as labels and fluorescence is developed after the assay is performed by adding an enhancement ligand to disassociate the chelated ions and creation of a new, fluorescent chelates in solution. Examples of chelate structures used for dissociative enhancement TRF assays include N′(pisothiocyanatobenzyl)diethylenetriamine, N¹, N², N³, N³-tetrakis(acetate), DPTA derivatives, [4-(p-isothiocyanatophenylethyl)pyridine-2,6-bis(methylenenitrilo)tetrakis (acetate) (Hemmila and Mukkala, supra). Examples of fluorescence enhancing ligands include thenoyltrifluoroacetone, TTA, β-napththoytrifluoroacetone, hexafluoroacetylacetone, and 2,4,6-trimethoxyphenyl-dipicolinic acid. An example of a dissociated TRF assay is marketed under the trademark DELFIA™ and is one of several TRF assays marketed to evaluate ligand-receptor interactions.

Fluorescent assays may be nondissociative enhancement assays. These assays use chelates with some energy absorbing properties; however, they need some enhancement. Other fluorescent assays use stable fluorescent chelates such as pyridine analogues, DPTA conjugates, polymacrocyclic cage-type complexes, such as cryptates, or calixarenes, podants, helicates, cyclic Schiff's bases, and others (Hemmila and Mukkala, supra. These chelates are used without enhancement and ion saturation; however, others may require various kinds of enhancement or ion saturation to enhance quantum yield. An example of a stable fluorescent chelates is 4-(p-isothiocyanatophenylethynyl)pyridine as tetra(acetate); others are described in Hemmila and Mukkala supra, Moore et al., supra; and, U.S. Pat. No. 5,859,215.

Luminescent chelates are used in a number of bioanalytical applications, including receptor ligand binding assays. Europium chelates are used as donors in TR-FRET assays, commercialized as TRACE/Kryptor® by Brahms Diagnostica, HTRF® by CisBio International, DELFIA and LANCE® by Perkin Elmer and LRET® by Amersham (Hemilla and Laitala supra). Lanthanide labels are attractive alternative to radiolabels because they are highly sensitive, can be automated, and can be used in multilabel studies.

With the appropriate use of linker groups in the case of acceptor fluorophores or chelates in the case of donor fluorophores, various acceptor or donor fluorophores can be used in labeling A₁ adenosine receptor compounds of formula (I) for use in a heterogeneous or homogeneous TRF assays. See for example, Baker et al., supra, Hemmila and Mukkala supra, Moore et al., supra, Handl and Gillies supra, and Hemilla and Laitala supra. For example, a compound of formula (I) of the invention may be labeled with a luminescent lanthanide chelate in the manner described in U.S. Pat. No. 5,859,215. Compounds of formula (I) may be labeled (e,g, conjugated) with a fluorophore label, chelate, or linker group to any functional group on R₁ through R₄ the compounds, e.g. with the use of N-hydroxysuccinimide ester via an amine group, a maleimide via a thiol group, or a hydrazide with an aldehyde group on the compound which are well known in the art. For example, referring to the compounds of formula (I) as defined herein, a fluorophore label may be bonded to one or more of the hydroxy groups, amino groups or carboxyl groups present such as at the R₁ through R₄ positions on the compound. Additionally, a fluorophore label may be bonded to one or more of the hydroxyl groups that may be present at the R₁ through R₄ positions on the compound. The fluorophore-labeled probes may be detected through appropriate and known analytical techniques

Diagnostic probes described above may be used in vivo or in situ for imaging. Furthermore, the diagnostic probes described above may be used for autoradiography. When used for in vivo imaging, these diagnostic probes can be formulated and combined with the appropriate pharmaceutical carrier for use as radiopharmaceuticals in a similar manner to that described for other pharmaceuticals of compounds of Formula (I) as previously described and incorporated as reference. See U.S. Pat. No. 7,247,639. Furthermore, the diagnostic probes, radiopharmaceuticals described above for compounds of Formula (I) may include salts, solvates, hydrates, and prodrugs for compounds of Formula (I) as previously described Furthermore the dosage for each formulation of the radiopharmaceutical in general is determined as a function of the amount of radioactivity needed for the diagnostic study and the condition of the patient, is expressed in becquerels or curies, and may vary for example from 5 microcuries to 35 millicuries.

The invention is also directed to radiopharmaceutical compositions which include compounds of the present invention and a pharmaceutically acceptable carrier. The radiopharmaceutical compositions described herein can be prepared by any applicable method of the art. The radiopharmaceutical composition is particularly useful in applications relating to organ preservation in vivo or in situ, perfusion of an isolated organ either removed or contained within the body (e.g., when an organ is transported for transplantation), cardiopulmonary bypass, perfusion of an extremity or limb, and the like. The compounds may be used in intra-articular, intra-thecal, gastrointestinal, and genital urinary applications, as well as in any cavity or lumen such as, for example, the thoracic cavity, or ear canal.

While the present invention is intended primarily for the imaging of human subjects, it will be appreciated that other subjects, particularly mammalian subjects such as dogs, cats, horses, rabbits, etc., can also be imaged by the methods of the present invention for veterinary purposes

The radiopharmaceutical compositions may be employed, as an example, in oral dosage form as a liquid composition. Such liquid compositions can include suspension compositions or syrup compositions and can be prepared with such carriers as water; a saccharide such as sucrose, sorbitol, fructose, and the like; a glycol such as polyethyleneglycol, polypropyleneglycol, and the like; an oil such as sesame oil, olive oil, soybean oil, and the like; an antiseptic such as p-hydroxy-benzoic acid esters and the like; and a flavor component such as a fruit flavor or a mint flavor.

The radiopharmaceutical compositions may also be in the form of powder, tablets, capsules, and tablets and can be prepared with various carriers. Suitable carriers include, but are not limited to, lactose, glucose, sucrose, mannitol, and the like; disintegrators such as starch, sodium alginate, and the like; binders such as polyvinyl alcohol, hydroxypropyl cellulose, gelatin, and the like; surfactants such as, for example, fatty acid esters; and plasticizers such as, for example, glycerins. The composition of the present invention is especially useful when applied sublingually. It should be noted that in the preparation of the tablets and capsules, a solid pharmaceutical carrier is used. Advantageously, the radiopharmaceutical compositions may be used in the form of, for example, eye drops or an aerosol.

Other types of radiopharmaceutical compositions may be employed in the form of a suppository, a nasal spray, and an injectable solution. These compositions are prepared using appropriate aqueous solutions which may include, but are not limited to, distilled water, and saline and buffer additives. Other components may be employed such as organic materials including neutral fatty bases. Additionally, the radiopharmaceutical compositions may be utilized in a transdermal application.

Biopolymers may be used as carriers in the above radiopharmaceutical compositions. Exemplary biopolymers may include, for example, proteins, sugars, lipids, or glycolipids. See, e.g., PCT Application WO 02/095391 (Published Nov. 22, 2002).

The present invention is explained in greater detail in the following non-limiting Examples.

### Example 1

### Synthesis of 5,6-Diamino-1-[2-(4-nitrophenyl)ethyl]-3-propyluracil (6)

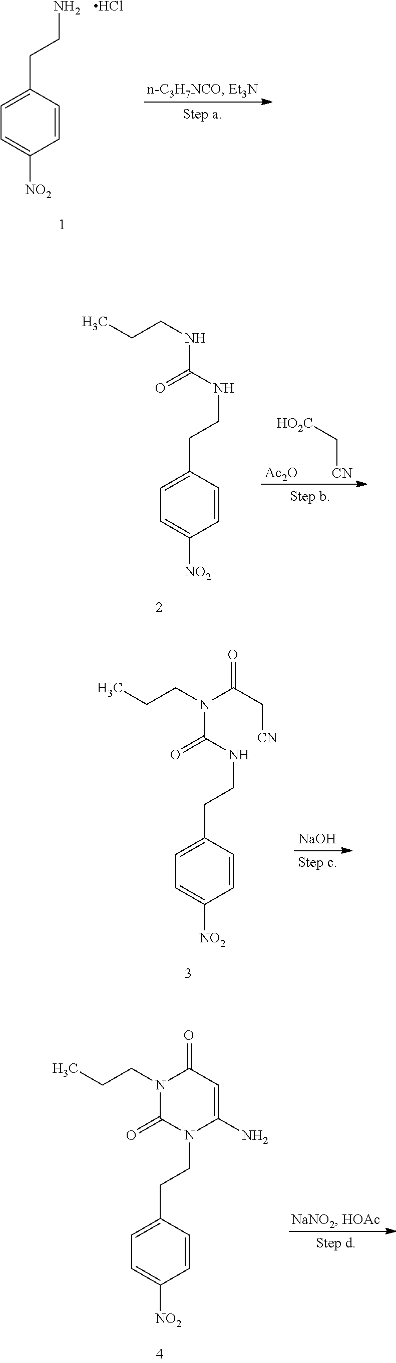

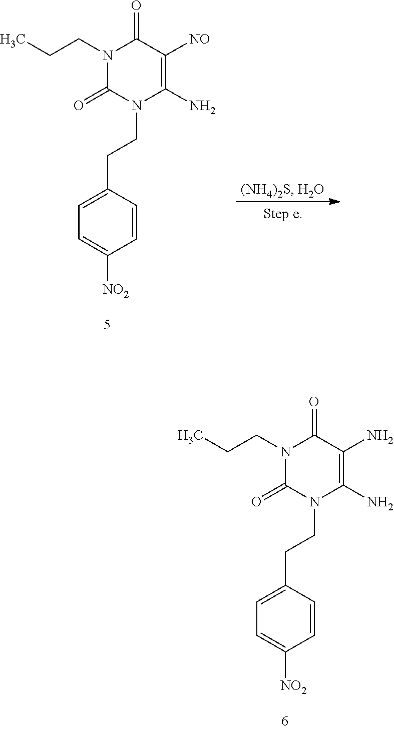

### Step a: Conversion of 4-Nitrophenethylamine Hydrochloride (1) to 1-[2-(4-Nitrophenyl)ethyl]-1′-propylurea (2)

To a slurry of 777 gm of 4-nitrophenethylamine hydrochloride (1) and 11.2 L of toluene was added slowly, 620 mL of triethylamine and this mixture was stirred for 30 min. at room temperature. To this suspension was then added slowly, 398 mL of n-propyl isocyanate, and the mixture was stirred overnight at room temperature to give a solid precipitate. The heterogeneous mixture was filtered and the isolated solids were washed with 1.5 L of toluene and then air dried. The 2.3 kg of crude product was stirred with 6 L of water to dissolve residual triethylamine hydrochloride. The solids were isolated by filtration and air dried. This material was dissolved in 4 L of absolute ethanol and 1 L of water was added to induce crystallization. The solids were filtered, washed with 2 L of 1:1 ethanol-water and air dried to yield a first crop of 880 gm of 1-[2-(4-nitrophenyl)ethyl]-1′-propylurea (2). The recrystallization mother liquors yielded an additional 39.8 gm of 1-[2-(4-nitrophenyl)ethyl]-1′-propylurea (2).

### Step b: Conversion of 1-[2-(4-Nitrophenyl)ethyl]-1′-propylurea (2) to 1-Cyanoacetyl-1-[2-(4-nitrophenyl)ethyl]-1′-propylurea (3)

A thick mixture of 920 gm of 1-[2-(4-nitrophenyl)ethyl]-1′-propylurea (2) and 1.0 L of acetic anhydride was stirred and warmed to ca. 50 degrees C. To this mixture was added 343.2 gm of cyanoacetic acid and 0.5 L of acetic anhydride and this homogeneous mixture was stirred at 80-85 degrees C. for three hours. The mixture was cooled and concentrated under vacuum to remove acetic acid and residual acetic anhydride. The residue was triturated successively with 1.0 L portions of water, acetonitrile, toluene and ethyl acetate. The residue was then dried under vacuum to yield 1261 gm of a 2:1 mixture of 1′-cyanoacetyl-1-[2-(4-nitrophenyl)ethyl]-1′-propylurea (3) and its undesired isomer 1-cyanoacetyl-1-[2-(4-nitrophenyl)ethyl]-1′-propylurea. This material was dissolved in 2.2 L of hot ethyl acetate to which ca. 750 mL of hexanes were added to the cloud point and the mixture was allowed to cool to room temperature to induce crystallization. Filtration of the solid and air drying yielded 363 gm of 1′-cyanoacetyl-1-[2-(4-nitrophenyl)ethyl]-1′-propylurea (3). If needed, additional recrystallizations from ethyl acetate-hexanes could be carried out to provide pure 1′-cyanoacetyl-1-[2-(4-nitrophenyl)ethyl]-1′-propylurea (3).

### Step c: Conversion of 1′-Cyanoacetyl-1-[2-(4-nitrophenyl)ethyl]-1′-propylurea (3) to 6-Amino-1-[2-(4-nitrophenyl)ethyl]-3-propyluracil (4)

A mixture of ca. 2N sodium hydroxide was produced by dissolving 336 gm of solid sodium hydroxide in 4.2 L of water. To this warm solution was added, in portions, 312 gm of 1′-cyanoacetyl-1-[2-(4-nitrophenyl)ethyl]-1′-propylurea (3) and the mixture was stirred for 1 hour at 80 degrees C., then was cooled to room temperature with stirring to induce crystallization. The solids were isolated by filtration, washed with four 500 mL portions of water and vacuum dried at 65 degrees C. to yield 232 gm of crude 6-amino-1-[2-(4-nitrophenyl)ethyl]-3-propyluracil (4).

### Step d: Conversion of 6-Amino-1-[2-(4-nitrophenyl)ethyl]-3-propyluracil (4) to 6-Amino-5-nitroso-1-[2-(4-nitrophenyl)ethyl]-3-propyl uracil (5)

To a solution of 232 gm of crude 6-amino-1-[2-(4-nitrophenyl)ethyl]-3-propyluracil (4), 4.0 L of water and ca. 2.0 L of ethanol at 80 degrees C. was added 55.3 gm of sodium nitrite in one portion, followed by the dropwise addition of 100 mL of glacial acetic acid. After stirring at 80 degrees C. for 20 minutes the mixture was allowed to cool to near room temperature, then was chilled in an ice bath to effect crystallization. The solids were isolated by filtration, washed with two 1.0 L portions of water and dried under vacuum to yield 244 gm of purple colored 6-amino-5-nitroso-1-[2-(4-nitrophenyl)ethyl]-3-propyluracil (5).

### Step e: Conversion of 6-Amino-5-nitroso-1-[2-(4-nitrophenyl)ethyl]-3-propyluracil (5) to 5,6-Diamino-1-[2-(4-nitrophenyl)ethyl]-3-propyluracil (6)

A mixture of 243 gm of 6-amino-5-nitroso-1-[2-(4-nitrophenyl)ethyl]-3-propyluracil (5), and 2.1 L of water was heated to reflux and 528 mL of a 50% aqueous solution of ammonium sulfide was added with stirring to control foaming. The dark solution was stirred at 90-100 degrees C. for 30 min. and allowed to cool with stirring for 1.5 hours. The mixture was then chilled in an ice bath to fully effect crystallization. The solids were isolated by filtration, washed with three 500 mL portions of water and dried under vacuum to yield 219 gm of a dark solid. This material was recrystallized from 1.0 L of acetonitrile to yield two crops totaling 169.5 gm of 5,6-diamino-1-[2-(4-nitrophenyl)ethyl]-3-propyluracil (6).

### Example 2

### Synthesis of 8-Benzyl-3-[2-(4-nitrophenyl)ethyl]-1-propylxanthine (9)

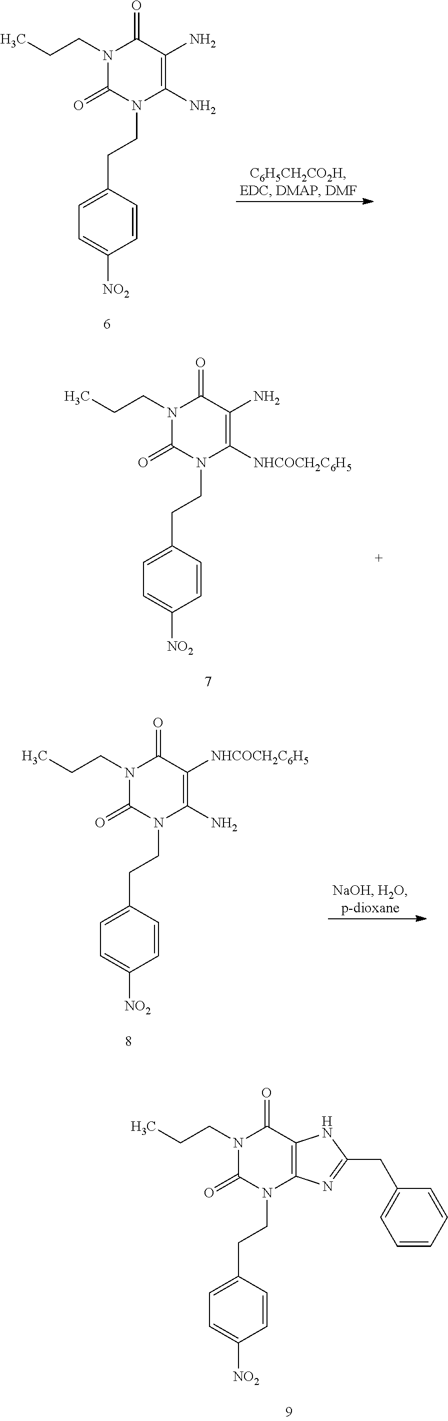

A solution of 44.9 gm of phenylacetic acid in 630 mL of dimethylformamide (DMF) was chilled in an ice water bath and 63.38 gm of 1-(3-dimethylaminopropyl)-3-ethylcarbodiimide hydrochloride (EDC) was added followed by 5.24 gm of 4-dimethylaminopyridine (DMAP). This mixture was stirred at ca. 4 degrees C. for 30 minutes and 100 gm of 5,6-diamino-1-[2-(4-nitrophenyl)ethyl]-3-propyluracil (6) was added in one portion. This mixture was stirred for 60 hr at room temperature. The dark homogeneous solution was poured into 700 mL of ice water with stirring to effect precipitation. The solids were isolated by filtration, washed with three 100 mL portions of water and dried under vacuum to yield 103 gm of a mixture of 5-amino-1-[2-(4-nitrophenyl)ethyl]-6-phenacetoamino-3-propyluracil (7) and 6-amino-1-[2-(4-nitrophenyl)ethyl]-5-phenacetoamino-3-propyluracil (8) intermediates. These solids were dissolved in 450 mL of p-dioxane, 600 mL of 2N aqueous sodium hydroxide was added and the mixture was heated at reflux for one hr. The solution was then chilled in an ice water bath and the pH adjusted to pH 4 with ca. 100 mL of concentrated hydrochloric acid to yield a precipitate. The solids were isolated by filtration, washed with three 100 mL portions of water and dried under vacuum to yield 82 gm of an orange solid. Recrystallization from hot ethyl acetate afforded 58.0 gm of 8-benzyl-3-[2-(4-nitrophenyl)ethyl]-1-propylxanthine (9).

### Example 3

### Synthesis of 8-Benzyl-7-[2-ethyl(2-hydroxyethyl)amino]ethyl-3-[2-(4-nitrophenyl)ethyl]-1-propylxanthine

A mixture of 2.1 gm of 8-benzyl-3-[2-(4-nitrophenyl)ethyl]-1-propylxanthine (9), 1.02 gm of sodium carbonate, 3.82 ml of 1,2-dichloroethane and 0.59 ml of 2-(ethylamino)ethanol was heated in a steel pressure vessel under argon at 120 degrees C. for 3-5 hours*. The mixture was then cooled and vented to the atmosphere. The semisolid reaction mixture was triturated several times with 5-10 ml portions of methanol followed by methylene chloride and the combined solutions were evaporated to dryness. The residue was purified by column chromatography on silica gel using a gradient of 1:1 ethyl acetate-hexanes, ethyl acetate and 5% methanol in ethyl acetate. The appropriate fractions were collected and evaporated to dryness to yield a light orange solid of 8-benzyl-7-[2-ethyl(2-hydroxyethyl)amino]ethyl-3-[2-(4-nitrophenyl)ethyl]-1-propylxanthine.

### Example 4

### Synthesis of 3-[2-(4-Aminophenyl)ethyl]-8-benzyl-7-[2-ethyl(2-hydroxyethyl)-amino]ethyl-1-propylxanthine Free Base and 3-[2-(4-Aminophenyl)ethyl]-8-benzyl-7-[2-ethyl(2-hydroxyethyl)-amino]ethyl-1-propylxanthine Dihydrochloride Salt

To a mixture of 9.4 gm of 8-benzyl-7-[2-ethyl(2-hydroxyethyl)amino]ethyl-3-[2-(4-nitrophenyl)ethyl]-1-propylxanthine in 400 ml of tetrahydrofuran under inert gas was added 1.2 gm of 10% palladium on carbon catalyst followed by the dropwise addition of 12 ml of hydrazine hydrate. The mixture was stirred for 2 hours at which time gas evolution subsided. An additional 600 mg of 10% palladium on carbon catalyst was added, followed by 5 ml of additional hydrazine hydrate. Additional catalyst and hydrazine hydrate were added as needed to complete the reaction. The reaction mixture was then filtered through Celite and evaporated to dryness to yield an orange oil. Purification by silica gel column chromatography afforded purified solid 3-[2-(4-aminophenyl)ethyl]-8-benzyl-7-[2-ethyl(2-hydroxyethyl)-amino]ethyl-1-propylxanthine free base which was dissolved in 75 ml of tetrahydrofuran. To this solution was added 15 ml of 4N hydrogen chloride in p-dioxane, which gave a white precipitate. This precipitate was stirred as a slurry, collected by filtration and vacuum dried to afford 3-[2-(4-aminophenyl)ethyl]-8-benzyl-7-[2-ethyl(2-hydroxyethyl)-amino]ethyl-1-propylxanthine dihydrochloride salt, m.p. 230-231 degrees C. (uncorrected).

### Example 5

### Synthesis of 3-[2-(4-Aminophenyl)ethyl]-8-benzyl-7-(2-ethylamino)ethyl-1-propylxanthine Free Base or Hydrochloride Salts

By the method of Example 3,8-benzyl-3-[2-(4-nitrophenyl)ethyl]-1-propylxanthine, is reacted with sodium carbonate, 1,2-dichloroethane and ethylamine to yield 8-benzyl-7-(2-ethylamino)ethyl-3-[2-(4-nitrophenyl)ethyl]-1-propylxanthine. By the method of Example 4,8-benzyl-7-(2-ethylamino)ethyl-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-8-benzyl-7-(2-ethylamino)ethyl-1-propylxanthine free base. The corresponding dihydrochloride salt is then made on exposure to an excess of hydrogen chloride in solution.

### Example 6

### Synthesis of 3-[2-(4-Aminophenyl)ethyl]-8-benzyl-7-(2,2-diethylamino)ethyl-1-propylxanthine Free Base or Hydrochloride Salts

By the method of Example 3,8-benzyl-3-[2-(4-nitrophenyl)ethyl]-1-propylxanthine (9), is reacted with sodium carbonate, 1,2-dichloroethane and diethylamine to yield 8-benzyl-7-(2,2-diethylamino)ethyl-3-[2-(4-nitrophenyl)ethyl]-1-propylxanthine. By the method of Example 4,8-benzyl-7-(2,2-diethylamino)ethyl-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-8-benzyl-7-(2,2-diethylamino)ethyl-1-propylxanthine free base. The corresponding dihydrochloride salt is then made on exposure to an excess of hydrogen chloride in solution.

### Example 7

### Synthesis of 3-[2-(4-Aminophenyl)ethyl]-7-[2-ethyl(2-hydroxyethyl)amino]ethyl-8-(4-fluorobenzyl)-1-propylxanthine Free Base or Hydrochloride Salts

By the method of Example 2,4-fluorophenylacetic acid is reacted with 5,6-diamino-1-[2-(4-nitrophenyl)ethyl]-3-propyluracil (6) to yield 8-(4-fluorobenzyl)-3-[2-(4-nitrophenyl)ethyl]-1-propylxanthine. By the methods of Example 3 and Example 4, 8-(4-fluorobenzyl)-3-[2-(4-nitrophenyl)ethyl]-1-propylxanthine is alkylated with a mixture of 1,2-dichloroethane and 2-(ethylamino)ethanol, to afford 7-[2-ethyl(2-hydroxyethyl)amino]ethyl-8-(4-fluorobenzyl-3-[2-(4-nitrophenyl)ethyl]-1-propylxanthine, which, in turn, is reduced with hydrazine hydrate or hydrogen gas in the presence of a palladium catalyst to yield 3-[2-(4-aminophenyl)ethyl]-7-[2-ethyl(2-hydroxyethyl)amino]ethyl-8-(4-fluorobenzyl)-1-propylxanthine free base. The corresponding dihydrochloride salt is then made on exposure to an excess of hydrogen chloride in solution.

### Example 8

### Synthesis of 3-[2-(4-Aminophenyl)ethyl]-7-[2-ethyl(2-hydroxyethyl)amino]ethyl-1-propyl-8-[(3-pyridyl)methyl]xanthine Free Base or Hydrochloride Salts

By the method of Example 2,3-pyridylacetic acid is reacted with 5,6-diamino-1-[2-(4-nitrophenyl)ethyl]-3-propyluracil (6) to yield 3-[2-(4-nitrophenyl)ethyl]-1-propyl-8-[(3-pyridyl)methyl]xanthine. By the method of Example 3, this substance is alkylated with a mixture of 1,2-dichloroethane and 2-(ethylamino)ethanol to yield 7-[2-ethyl(2-hydroxyethyl)amino]ethyl-3-[2-(4-nitrophenyl)ethyl]-1-propyl-8-[(3-pyridyl)methyl]xanthine. By the method of Example 4 this substance is reduced with hydrazine hydrate or hydrogen gas in the presence of a palladium catalyst to yield 3-[2-(4-aminophenyl)ethyl]-7-[2-ethyl(2-hydroxyethyl)amino]ethyl-1-propyl-8-[(3-pyridyl)methyl]xanthine free base. The corresponding hydrochloride salt is then made on exposure to an excess of hydrogen chloride in solution.

### Example 9

### Synthesis of 3-[2-(4-Aminophenyl)ethyl]-7-(2-ethylamino)ethyl-1-propyl-8-[(3-pyridyl)methyl]xanthine Free Base or Hydrochloride Salts

By the method of Example 2,3-pyridylacetic acid is reacted with 5,6-diamino-1-[2-(4-nitrophenyl)ethyl]-3-propyluracil (6) to yield 3-[2-(4-nitrophenyl)ethyl]-1-propyl-8-[(3-pyridyl)methyl]xanthine. By the method of Example 3, this substance is reacted with sodium carbonate, 1,2-dichloroethane and ethylamine to yield 4,7-(2-ethylamino)ethyl-3-[2-(4-nitrophenyl)ethyl]-1-propyl-8-[(3-pyridyl)methyl]xanthine. By the method of Example 4,7-(2-ethylamino)ethyl-3-[2-(4-nitrophenyl)ethyl]-1-propyl-8-[(3-pyridyl)methyl]xanthine is reduced with hydrazine hydrate or hydrogen gas in the presence of a palladium catalyst to yield 3-[2-(4-aminophenyl)ethyl]-7-(2-ethylamino)ethyl-1-propyl-8-[(3-pyridyl)methyl]xanthine free base. The corresponding hydrochloride salt is then made on exposure to an excess of hydrogen chloride in solution.

### Example 10

### Synthesis of 3-[2-(4-Aminophenyl)ethyl]-7-(2,2-diethylamino)ethyl-1-propyl-8-[(3-pyridyl)methyl]xanthine Free Base or Hydrochloride Salts

By the method of Example 3, 3-[2-(4-nitrophenyl)ethyl]-1-propyl-8-[(3-pyridyl)methyl]xanthine, is reacted with sodium carbonate, 1,2-dichloroethane and diethylamine to yield 7-(2,2-diethylamino)ethyl-3-[2-(4-nitrophenyl)ethyl]-1-propyl-8-[(3-pyridyl)methyl]xanthine. By the method of Example 4,7-(2,2-diethylamino)ethyl-3-[2-(4-nitrophenyl)ethyl]-1-propyl-8-[(3-pyridyl)methyl]xanthine is reduced with hydrazine hydrate or hydrogen gas in the presence of a palladium catalyst to yield 3-[2-(4-aminophenyl)ethyl]-7-(2,2-diethylamino)ethyl-8-1-propyl-8-[(3-pyridyl)methyl]xanthine free base. The corresponding hydrochloride salt is then made on exposure to an excess of hydrogen chloride in solution.

### Example 11

### Synthesis of 3-[2-(4-Aminophenyl)ethyl]-7-[2-ethyl(2-hydroxyethyl)amino]ethyl-1-propyl-8-[(thiophen-2-yl)methyl]xanthine Free Base or Hydrochloride Salts

By the method of Example 2,2-thiopheneacetic acid is reacted with 5,6-diamino-1-[2-(4-nitrophenyl)ethyl]-3-propyluracil (6) to yield 3-[2-(4-nitrophenyl)ethyl]-1-propyl-8-[(thiophen-2-yl)methyl]xanthine. By the method of Example 3, this substance is alkylated with a mixture of 1,2-dichloroethane, sodium carbonate and 2-(ethylamino)ethanol to yield 7-[2-ethyl(2-hydroxyethyl)amino]ethyl-3-[2-(4-nitrophenyl)ethyl]-1-propyl-8-[(thiophen-2-yl)methyl]xanthine. By the method of Example 4 this substance is reduced with hydrazine hydrate or hydrogen gas in the presence of a palladium catalyst to yield 3-[2-(4-aminophenyl)ethyl]-7-[2-ethyl(2-hydroxyethyl)amino]ethyl-1-propyl-8-[(thiophen-2-yl)methyl]xanthine free base. The corresponding dihydrochloride salt is then made on exposure to an excess of hydrogen chloride in solution.

### Example 12

### Synthesis of 3-[2-(4-Aminophenyl)ethyl]-7-[2-ethyl(2-hydroxyethyl)amino]ethyl-1-propyl-8-[(4-thiazolyl)methyl]xanthine Free Base or Hydrochloride Salts

By the method of Example 2,4-thiazolylacetic acid is reacted with 5,6-diamino-1-[2-(4-nitrophenyl)ethyl]-3-propyluracil (6) to yield 3-[2-(4-nitrophenyl)ethyl]-1-propyl-8-[(4-thiazolyl)methyl]xanthine. By the method of Example 3, this substance is alkylated with a mixture of 1,2-dichloroethane, sodium carbonate and 2-(ethylamino)ethanol to yield 7-[2-ethyl(2-hydroxyethyl)amino]ethyl-3-[2-(4-nitrophenyl)ethyl]-1-propyl-8-[(4-thiazolyl)methyl]xanthine. By the method of Example 4 this substance is reduced with hydrazine hydrate or hydrogen gas in the presence of a palladium catalyst to yield 3-[2-(4-aminophenyl)ethyl]-7-[2-ethyl(2-hydroxyethyl)amino]ethyl-1-propyl-8-[(4-thiazolyl)methyl]xanthine free base. The corresponding hydrochloride salt is then made on exposure to an excess of hydrogen chloride in solution.

### Example 13

### Synthesis of 3-[2-(4-Aminophenyl)ethyl]-7-[2-ethyl(2-hydroxyethyl)amino]ethyl-1-propyl-8-[(1H-tetrazol-5-yl)methyl]xanthine Free Base or Hydrochloride Salts

By the method of Example 2, 1H-tetrazole-5-acetic acid is reacted with 5,6-diamino-1-[2-(4-nitrophenyl)ethyl]-3-propyluracil (6) to yield 3-[2-(4-nitrophenyl)ethyl]-1-propyl-8-[(1H-tetrazol-5-yl)methyl]xanthine. By the method of Example 3, this substance is alkylated with a mixture of 1,2-dichloroethane, sodium carbonate and 2-(ethylamino)ethanol to yield 7-[2-ethyl(2-hydroxyethyl)amino]ethyl-3-[2-(4-nitrophenyl)ethyl]-1-propyl-8-[(1H-tetrazol-5-yl)methyl]xanthine. By the method of Example 4 this substance is reduced with hydrazine hydrate or hydrogen gas in the presence of a palladium catalyst to yield 3-[2-(4-aminophenyl)ethyl]-7-[2-ethyl(2-hydroxyethyl)amino]ethyl-1-propyl-8-[(1H-tetrazol-5-yl)methyl]xanthine free base. The corresponding hydrochloride salt is then made on exposure to an excess of hydrogen chloride in solution.

### Example 14

### Synthesis of 3-[2-(4-Aminophenyl)ethyl]-7-(2-ethylamino)ethyl-1-propyl-8-[(1H-tetrazol-5-yl)methyl]xanthine Free Base or Hydrochloride Salts

By the method of Example 2, 1 H-tetrazole-5-acetic acid is reacted with 5,6-diamino-1-[2-(4-nitrophenyl)ethyl]-3-propyluracil (6) to yield 3-[2-(4-nitrophenyl)ethyl]-1-propyl-8-[(1H-tetrazol-5-yl)methyl]xanthine. By the method of Example 3, this substance is alkylated with a mixture of 1,2-dichloroethane, sodium carbonate, and ethylamine to yield 7-(2-ethylamino)ethyl-3-[2-(4-nitrophenyl)ethyl]-1-propyl-8-[(1H-tetrazol-5-yl)methyl]xanthine. By the method of Example 4,7-(2-ethylamino)ethyl-3-[2-(4-nitrophenyl)ethyl]-1-propyl-8-[(1H-tetrazol-5-yl)methyl]xanthine is reduced with hydrazine hydrate or hydrogen gas in the presence of a palladium catalyst to yield 3-[2-(4-aminophenyl)ethyl]-7-(2-ethylamino)ethyl-1-propyl-8-[(1H-tetrazol-5-yl)methyl]xanthine free base. The corresponding hydrochloride salt is then made on exposure to an excess of hydrogen chloride in solution.

### Example 15

### Synthesis of 3-[2-(4-Aminophenyl)ethyl]-7-(2,2-diethylamino)ethyl-1-propyl-8-[(1H-tetrazol-5-yl)methyl]xanthine Free Base or Hydrochloride Salts

By the method of Example 3, 3-[2-(4-nitrophenyl)ethyl]-1-propyl-8-[(1H-tetrazol-5-yl)methyl]xanthine, is reacted with sodium carbonate, 1,2-dichloroethane and diethylamine to yield 7-(2,2-diethylamino)ethyl-3-[2-(4-nitrophenyl)ethyl]-1-propyl-8-[(1H-tetrazol-5-yl)methyl]xanthine. By the method of Example 4, 7-(2,2-diethylamino)ethyl-3-[2-(4-nitrophenyl)ethyl]-1-propyl-8-[(1H-tetrazol-5-yl)methyl]xanthine is reduced with hydrazine hydrate or hydrogen gas in the presence of a palladium catalyst to yield 3-[2-(4-aminophenyl)ethyl]-7-(2,2-diethylamino)ethyl-8-1-propyl-8-[(1H-tetrazol-5-yl)methyl]xanthine free base. The corresponding hydrochloride salt is then made on exposure to an excess of hydrogen chloride in solution.

### Example 16

### Synthesis of 3-[2-(4-Aminophenyl)ethyl]-7-[2-ethyl(2-hydroxyethyl)amino]ethyl-8-(4-methylsulfonobenzyl)-1-propylxanthine Free Base or Hydrochloride Salts

By the method of Example 2,4-methylsulfonophenylacetic acid is reacted with 5,6-diamino-1-[2-(4-nitrophenyl)ethyl]-3-propyluracil (6) to yield 3-[2-(4-nitrophenyl)ethyl]-8-(4-methylsulfonobenzyl)-1-propylxanthine. By the method of Example 3, 3-[2-(4-nitrophenyl)ethyl]-8-(4-methylsulfonobenzyl)-1-propylxanthine, is reacted with sodium carbonate, 1,2-dichloroethane and 2-(ethylamino)ethanol to yield 7-[2-ethyl(2-hydroxyethyl)amino]ethyl-8-(4-methylsulfonobenzyl)-3-[2-(4-nitrophenyl)ethyl]-1-propylxanthine. By the method of Example 4, 7-[2-ethyl(2-hydroxyethyl)amino]ethyl-8-(4-methylsulfonobenzyl)-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-7-[2-ethyl(2-hydroxyethyl)amino]ethyl-8-(4-methylsulfonobenzyl)-1-propylxanthine free base. The corresponding hydrochloride salt is then made on exposure to an excess of hydrogen chloride in solution.

### Example 17

### Synthesis of 3-[2-(4-Aminophenyl)ethyl]-7-(2-ethylamino)ethyl-8-(4-methylsulfonobenzyl)-1-propylxanthine Free Base or Hydrochloride Salts

By the method of Example 2,4-methylsulfonophenylacetic acid is reacted with 5,6-diamino-1-[2-(4-nitrophenyl)ethyl]-3-propyluracil (6) to yield 3-[2-(4-nitrophenyl)ethyl]-8-(4-methylsulfonobenzyl)-1-propylxanthine. By the method of Example 3, 3-[2-(4-nitrophenyl)ethyl]-8-(4-methylsulfonobenzyl)-1-propylxanthine, is reacted with sodium carbonate, 1,2-dichloroethane and ethylamine to yield 7-(2-ethylamino)ethyl-8-(4-methylsulfonobenzyl)-3-[2-(4-nitrophenyl)ethyl]-1-propylxanthine. By the method of Example 4,7-(2-ethylamino)ethyl-8-(3-methylsulfonobenzyl)-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-7-(2-ethylamino)ethyl-8-(4-methylsulfonobenzyl)-1-propylxanthine free base. The corresponding hydrochloride salt is then made on exposure to an excess of hydrogen chloride in solution.

### Example 18

### Synthesis of 3-[2-(4-Aminophenyl)ethyl]-7-(2,2-diethylamino)ethyl-8-(4-methylsulfonobenzyl)-1-propylxanthine Free Base or Hydrochloride Salts

By the method of Example 2,4-methylsulfonophenylacetic acid is reacted with 5,6-diamino-1-[2-(4-nitrophenyl)ethyl]-3-propyluracil (6) to yield 3-[2-(4-nitrophenyl)ethyl]-1-8-(4-methylsulfonobenzyl)-1-propylxanthine. By the method of Example 3, is reacted with sodium carbonate, 1,2-dichloroethane and diethylamine to yield 7-(2,2-diethylamino)ethyl-8-(4-methylsulfonobenzyl)-3-[2-(4-nitrophenyl)ethyl]-1-propylxanthine. By the method of Example 4, 7-(2,2-diethylamino)ethyl-8-(4-methylsulfonobenzyl)-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-7-(2,2-diethylamino)ethyl-8-(4-methylsulfonobenzyl)-1-propylxanthine free base. The corresponding hydrochloride salt is then made on exposure to an excess of hydrogen chloride in solution.

### Example 19

### Synthesis of 3-[2-(4-Aminophenyl)ethyl]-8-benzyl-7-[2-ethyl(2-hydroxyethyl)amino]ethyl-1-(3-methoxypropyl)xanthine

By methods well known in the art 3-methoxypropyl isocyanate is converted into 8-benzyl-3-[2-(4-nitrophenyl)ethyl]-1-(3-methoxypropyl)xanthine. By the method of Example 3, this substance is alkylated with a mixture of 1,2-dichloroethane, sodium carbonate and 2-(ethylamino)ethanol to yield 8-benzyl-7-[2-ethyl(2-hydroxyethyl)amino]ethyl-3-[2-(4-nitrophenyl)ethyl]-1-(3-methoxypropyl)xanthine. By the method of Example 4 this substance is reduced with hydrazine hydrate or hydrogen gas in the presence of a palladium catalyst to yield 3-[2-(4-aminophenyl)ethyl]-8-benzyl-7-[2-ethyl(2-hydroxyethyl)amino]ethyl-1-(3-methoxypropyl)xanthine free base. The corresponding hydrochloride salt is then made on exposure to an excess of hydrogen chloride in solution.

### Example 20

### Synthesis of 3-[2-(4-Aminophenyl)ethyl]-8-benzyl-7-(2-ethylamino)ethyl-1-(3-methoxypropyl)xanthine

By methods well known in the art 3-methoxypropyl isocyanate is converted into 8-benzyl-3-[2-(4-nitrophenyl)ethyl]-1-(3-methoxypropyl)xanthine. By the method of Example 3, this substance is alkylated with a mixture of 1,2-dichloroethane, sodium carbonate and ethylamine to yield 8-benzyl-7-(2-ethylamino)ethyl-3-[2-(4-nitrophenyl)ethyl]-1-(3-methoxypropyl)xanthine. By the method of Example 4 this substance is reduced with hydrazine hydrate or hydrogen gas in the presence of a palladium catalyst to yield 3-[2-(4-aminophenyl)ethyl]-8-benzyl-7-(2-ethylamino)ethyl-1-(3-methoxypropyl)xanthine free base. The corresponding hydrochloride salt is then made on exposure to an excess of hydrogen chloride in solution.

### Example 21

### Synthesis of 3-[2-(4-Aminophenyl)ethyl]-8-benzyl-7-(2,2-diethylamino)ethyl-1-(3-methoxypropyl)xanthine

By methods well known in the art 3-methoxypropyl isocyanate is converted into 8-benzyl-3-[2-(4-nitrophenyl)ethyl]-1-(3-methoxypropyl)xanthine. By the method of Example 3, this substance is alkylated with a mixture of 1,2-dichloroethane, sodium carbonate and diethylamine to yield 8-benzyl-7-(2,2-diethylamino)ethyl-3-[2-(4-nitrophenyl)ethyl]-1-(3-methoxypropyl)xanthine. By the method of Example 4 this substance is reduced with hydrazine hydrate or hydrogen gas in the presence of a palladium catalyst to yield 3-[2-(4-aminophenyl)ethyl]-8-benzyl-7-(2,2-diethylamino)ethyl-1-(3-methoxypropyl)xanthine free base. The corresponding hydrochloride salt is then made on exposure to an excess of hydrogen chloride in solution.

### Example 22

### Synthesis of 3-[2-(4-Aminophenyl)ethyl]-8-benzyl-1-(3-dimethylaminopropyl)-7-[2-ethyl(2-hydroxyethyl)amino]ethylxanthine

By methods well known in the art 3-dimethylaminopropyl isocyanate is converted into 8-benzyl-3-[2-(4-nitrophenyl)ethyl]-1-(3-dimethylaminopropyl)xanthine. By the method of Example 3, this substance is alkylated with a mixture of 1,2-dichloroethane, sodium carbonate and 2-(ethylamino)ethanol to yield 8-benzyl-1-(3-dimethylaminopropyl)-7-[2-ethyl(2-hydroxyethyl)amino]ethyl-3-[2-(4-nitrophenyl)ethyl]xanthine. By the method of Example 4 this substance is reduced with hydrazine hydrate or hydrogen gas in the presence of a palladium catalyst to yield 3-[2-(4-aminophenyl)ethyl]-8-benzyl-1-(3-dimethylaminopropyl)-7-[2-ethyl(2-hydroxyethyl)amino]ethylxanthine free base. The corresponding hydrochloride salt is then made on exposure to an excess of hydrogen chloride in solution.

### Example 23

### Synthesis of 3-[2-(4-Aminophenyl)ethyl]-8-benzyl-1-(3-dimethylaminopropyl)-7-(2-ethylamino)ethylxanthine

By methods well known in the art 3-dimethylaminopropyl isocyanate is converted into 8-benzyl-3-[2-(4-nitrophenyl)ethyl]-1-(3-dimethylaminopropyl)xanthine. By the method of Example 3, this substance is alkylated with a mixture of 1,2-dichloroethane, sodium carbonate and ethylamine to yield 8-benzyl-1-(3-dimethylaminopropyl)-7-(2-ethylamino)ethyl-3-[2-(4-nitrophenyl)ethyl]xanthine. By the method of Example 4 this substance is reduced with hydrazine hydrate or hydrogen gas in the presence of a palladium catalyst to yield 3-[2-(4-aminophenyl)ethyl]-8-benzyl-1-(3-dimethylaminopropyl)-7-(2-ethylamino)ethylxanthine free base. The corresponding hydrochloride salt is then made on exposure to an excess of hydrogen chloride in solution.

### Example 24

### Synthesis of 3-[2-(4-Aminophenyl)ethyl]-8-benzyl-7-(2,2-diethylamino)ethyl-1-(3-dimethylaminopropyl)xanthine

By methods well known in the art 3-dimethylaminopropyl isocyanate is converted into 8-benzyl-3-[2-(4-nitrophenyl)ethyl]-1-(3-dimethylaminopropyl)xanthine. By the method of Example 3, this substance is alkylated with a mixture of 1,2-dichloroethane, sodium carbonate and diethylamine to yield 8-benzyl-1-(3-dimethylaminopropyl)-7-(2,2-diethylamino)ethyl-3-[2-(4-nitrophenyl)ethyl]xanthine. By the method of Example 4 this substance is reduced with hydrazine hydrate or hydrogen gas in the presence of a palladium catalyst to yield 3-[2-(4-aminophenyl)ethyl]-8-benzyl-7-(2,2-diethylamino)ethyl-1-(3-dimethylaminopropyl)xanthine free base. The corresponding hydrochloride salt is then made on exposure to an excess of hydrogen chloride in solution.

### Example 25

### Synthesis of 3-[2-[4-(6-Aminohexanoyl)aminophenyl]ethyl]-8-benzyl-7-(2,2-diethylamino)ethyl-1-propylxanthine

By methods well known in the art, 3-[2-(4-aminophenyl)ethyl]-8-benzyl-7-(2,2-diethylamino)ethyl-1-propylxanthine is reacted with 6-aminohexanoic acid and a coupling agent such as 1-(3-dimethylaminopropyl)-3-ethylcarbodiimide hydrochloride (EDC) to yield 3-[2-[4-(6-aminohexanoyl)aminophenyl]ethyl]-8-benzyl-7-(2,2-diethylamino)ethyl-1-propylxanthine.

### Example 26

### Synthesis of the Cy3B-Coupled Amido Derivative of 3-[2-[4-(6-Aminohexyl-3-amidocarboxypropanoyl)]aminophenyl]ethyl]-8-benzyl-7-(2,2-diethylamino]ethyl-1-propylxanthine

By methods well known in the art, 3-[2-[4-(6-aminohexyl-3-amidocarboxypropanoyl)]aminophenyl]ethyl]-8-benzyl-7-(2,2-diethylamino]ethyl-1-propylxanthine is reacted with the commercially available 6,7,9,10-tetrahydro-2-carboxymethyl-14-sulfonato-16,16,18,18-tetramethyl-7aH,8aH-bisindolinium[3,2-a,3′2′-a]pyrano[3,2-c;5,6-c′]dipyridin-5-ium, N-hydroxysuccinimidyl ester (sold as Cy3B by Amersham Biosciences UK Limited, Little Chalfont, Buckinghamshire, England) and a base such as diisopropylethylamine to yield the Cy3B-coupled amido derivative of 3-[2-[4-(6-aminohexyl-3-amidocarboxypropanoyl)]aminophenyl]ethyl]-8-benzyl-7-(2,2-diethylamino]ethyl-1-propylxanthine.

### Example 27

### Synthesis of 3-[2-[4-(6-Aminohexyl-3-amidocarboxypropanoyl)aminophenyl]ethyl]-8-benzyl-7-(2,2-diethylamino) ethyl-1-propylxanthine

By methods well known in the art, 3-[2-(4-aminophenyl)ethyl]-8-benzyl-7-(2,2-diethylamino)ethyl-1-propylxanthine is reacted with succinyl anhydride and a base such as triethylamine to yield 3-[2-[4-(3-carboxypropanoyl)aminophenyl]ethyl]-8-benzyl-7-(2,2-diethylamino)ethyl-1-propylxanthine. In turn, this substance is then reacted with 1,6-diaminohexane and a coupling agent such as 1-(3-dimethylaminopropyl)-3-ethylcarbodiimide hydrochloride (EDC) to yield 3-[2-[4-(6-aminohexyl-3-amidocarboxypropanoyl)]aminophenyl]ethyl]-8-benzyl-7-(2,2-diethylamino]ethyl-1-propylxanthine.

### Example 28

### Synthesis of the Cy3B-Coupled Amido Derivative of 3-[2-[4-(6-Aminohexanoyl)aminophenyl]ethyl]-8-benzyl-7-(2,2-diethylamino)ethyl-1-propylxanthine

This compound can be prepared in an analogous manner as that described in Example 26 using corresponding starting materials.

### Example 29

### Synthesis of the d-Biotin-Coupled Amido Derivative of 3-[2-[4-(6-Aminohexyl-3-amidocarboxypropanoyl)]aminophenyl]ethyl]-8-benzyl-7-(2,2-diethylamino]ethyl-1-propylxanthine

By methods well known in the art, 3-[2-[4-(6-aminohexyl-3-amidocarboxypropanoyl)]aminophenyl]ethyl]-8-benzyl-7-(2,2-diethylamino]ethyl-1-propylxanthine is reacted with d-biotin and a coupling agent such as 1-(3-dimethylaminopropyl)-3-ethylcarbodiimide hydrochloride (EDC) to yield the d-biotin-coupled amido derivative of 3-[2-[4-(6-aminohexyl-3-amidocarboxypropanoyl)]aminophenyl]ethyl]-8-benzyl-7-(2,2-diethylamino]ethyl-1-propylxanthine.

### Example 30

### Synthesis of Tritium Labeled 3-[2-(4-Aminophenyl)ethyl]-8-benzyl-7-[1³H,2³H-[2-ethyl(2-hydroxyethyl)amino]ethyl]-1-propylxanthine Free Base and 3-[2-(3-Aminophenyl)ethyl]-8-benzyl-7-[1³H,2³H-[2-ethyl(2-hydroxyethyl)amino]ethyl]-1-propylxanthine Dihydrochloride Salt

By the method of Example 3,8-benzyl-3-[2-(4-nitrophenyl)ethyl]-1-propylxanthine is alkylated with a mixture of tritium-labeled 1,2-dichloroethane[³H-1,2-dichloroethane] and 2-(ethylamino)ethanol to yield tritium-labeled 8-benzyl-7-[1³H,2³H[2-ethyl(2-hydroxyethyl)amino]ethyl]-3-[2-(4-nitrophenyl)ethyl]-1-propylxanthine. By the method of Example 4 this substance is reduced with hydrazine hydrate or hydrogen gas in the presence of a palladium catalyst to yield tritium-labeled 3-[2-(4-aminophenyl)ethyl]-8-benzyl-7-[1³H,2³H-[2-ethyl(2-hydroxyethyl)amino]ethyl]-1-propylxanthine free base. The corresponding tritium-labeled dihydrochloride salt is then made on exposure to an excess of hydrogen chloride in solution.

### Example 31

### Synthesis of Tritium Labelled 3-[2-(4-Aminophenyl)ethyl]-8-benzyl-7-[1³H,2³H-(2-ethylamino)ethyl]-1-propylxanthine Free Base or Hydrochloride Salts

By the method of Example 3,8-benzyl-3-[2-(4-nitrophenyl)ethyl]-1-propylxanthine, is reacted with sodium carbonate, tritium-labeled 1,2-dichloroethane [³H-1,2-dichloroethane] and ethylamine to yield 8-benzyl-7-[1³H,2³H-(2-ethylamino)ethyl]-3-[2-(4-nitrophenyl)ethyl]-1-propylxanthine. By the method of Example 4,8-benzyl-7-[1³H,2³H-(2-ethylamino)ethyl]-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-8-benzyl-7-[1³H,2³H-(2-ethylamino)ethyl]-1-propylxanthine free base. The corresponding dihydrochloride salt is then made on exposure to an excess of hydrogen chloride in solution.

### Example 32

### Synthesis of 3-[4-(4-Aminophenyl)butyl]-8-benzyl-7-[2-ethyl(2-hydroxyethyl)amino]ethyl-1-propylxanthine

By the method of Example 2, phenylacetic acid is reacted with 5,6-diamino-1-[4-(4-nitrophenyl)butyl]-3-propyluracil to yield 8-benzyl-1-propyl-3-[4-(4-nitrophenyl)butyl]xanthine. In turn, 5,6-diamino-3-propyl-1-[4-(4-nitroyphenyl)butyl]-3-uracil is made by the synthetic methods of Example 1, starting with n-propyl isocyanate and 4-(4-nitrophenyl)butylamine. By the methods of Example 3 and Example 4,8-benzyl-3-[4-(4-nitrophenyl)butyl]-1-propylxanthine is alkylated with a mixture of 1,2-dichloroethane and 2-(ethylamino)ethanol, to afford 8-benzyl-7-[2-ethyl(2-hydroxyethyl)amino]ethyl-3-[4-(4-nitrophenyl)butyl]-1-propylxanthine, which, in turn, is reduced with hydrazine hydrate or hydrogen gas in the presence of a palladium catalyst to yield 3-[4-(4-aminophenyl)butyl]-8-benzyl-7-[2-ethyl(2-hydroxyethyl)amino]ethyl-1-propylxanthine free base. The corresponding dihydrochloride salt is then made on exposure to an excess of hydrogen chloride in solution.

### Example 33

### Synthesis of 3-[4-(4-Aminophenyl)butyl]-7-[2-ethyl(2-hydroxyethyl)amino]ethyl-1-propyl-8-(4-sulfonoxybenzyl)xanthine

By the method of Example 2,4-sulfonoxyphenylacetic acid is reacted with 5,6-diamino-1-[4-(4-nitrophenyl)butyl]-3-propyluracil (6) to yield 3-[4-(4-nitrophenyl)butyl]-1-propyl-8-(4-sulfonoxybenzyl)xanthine. In turn, 5,6-diamino-1-[4-(4-nitrophenyl)butyl]-3-propyluracil is made by the synthetic methods of Example 1, starting with n-propyl isocyanate and 4-(4-nitrophenyl)butylamine. By the method of Example 3, 3-[4-(4-nitrophenyl)butyl]-1-propyl-8-(4-sulfonoxybenzyl)xanthine, is reacted with excess sodium carbonate, 1,2-dichloroethane and 2-(ethylamino)ethanol to yield, after aqueous work-up, 7-[2-ethyl(2-hydroxyethyl)amino]ethyl-3-[4-(4-nitrophenyl)butyl]-1-propyl-8-(4-sulfonoxybenzyl)xanthine. By the method of Example 4, 7-[2-ethyl(2-hydroxyethyl)amino]ethyl-3-[4-(4-nitrophenyl)ethyl]-1-propyl-8-(4-sulfonoxybenzyl)xanthine is reduced with hydrazine hydrate or hydrogen gas in the presence of a palladium catalyst to yield 3-[4-(4-aminophenyl)butyl]-7-[2-ethyl(2-hydroxyethyl)amino]ethyl-1-propyl-8-(4-sulfonoxybenzyl)xanthine.

### Example 34

### Synthesis of 3-[4-(4-Aminophenyl)butyl]-7-(2-ethylamino)ethyl-1-propyl-8-(4-sulfonoxybenzyl)xanthine

By the method of Example 2,4-sulfonoxyphenylacetic acid is reacted with 5,6-diamino-1-[4-(4-nitrophenyl)butyl]-3-propyluracil (6) to yield 3-[4-(4-nitrophenyl)butyl]-1-propyl-8-(4-sulfonoxybenzyl)xanthine. In turn, 5,6-diamino-1-[4-(4-nitrophenyl)butyl]-3-propyluracil is made by the synthetic methods of Example 1, starting with n-propyl isocyanate and 4-(4-nitrophenyl)butylamine. By the method of Example 3, 3-[4-(4-nitrophenyl)butyl]-1-propyl-8-(4-sulfonoxybenzyl)xanthine, is reacted with excess sodium carbonate, 1,2-dichloroethane and ethylamine to yield, after aqueous work-up, 7-(2-ethylamino)ethyl-3-[4-(4-nitrophenyl)butyl]-1-propyl-8-(4-sulfonoxybenzyl)xanthine. By the method of Example 4,7-(2-ethylamino)ethyl-3-[4-(4-nitrophenyl)butyl]-1-propyl-8-(4-sulfonoxybenzyl)xanthine is reduced with hydrazine hydrate or hydrogen gas in the presence of a palladium catalyst to yield 3-[4-(4-aminophenyl)butyl]-7-(2-ethylamino)ethyl-1-propyl-8-(4-sulfonoxybenzyl)xanthine.

### Example 35

### Synthesis of 3-[4-(4-Aminophenyl)butyl]-7-(2,2-diethylamino)ethyl-1-propyl-8-(4-sulfonoxybenzyl)xanthine

By the method of Example 2,4-sulfonoxyphenylacetic acid is reacted with 5,6-diamino-1-[4-(4-nitrophenyl)butyl]-3-propyluracil (6) to yield 3-[4-(4-nitrophenyl)butyl]-1-propyl-8-(4-sulfonoxybenzyl)xanthine. In turn, 5,6-diamino-1-[4-(4-nitrophenyl)butyl]-3-propyluracil is made by the synthetic methods of Example 1, starting with n-propyl isocyanate and 4-(4-nitrophenyl)butylamine. By the method of Example 3, 3-[4-(4-nitrophenyl)butyl]-1-propyl-8-(4-sulfonoxybenzyl)xanthine, is reacted with excess sodium carbonate, 1,2-dichloroethane and diethylamine to yield, after aqueous work-up, 7-(2,2-diethylamino)ethyl-3-[2-(4-nitrophenyl)butyl]-1-propyl-8-(4-sulfonoxybenzyl)xanthine. By the method of Example 4, 7-(2,2-diethylamino)ethyl-3-[4-(4-nitrophenyl)butyl]-1-propyl-8-(4-sulfonoxybenzyl)xanthine is reduced with hydrazine hydrate or hydrogen gas in the presence of a palladium catalyst to yield 3-[4-(4-aminophenyl)butyl]-7-(2,2-diethylamino)ethyl-1-propyl-8-(4-sulfonoxybenzyl)xanthine.

### Example 36

### Synthesis of 3-[4-(4-Aminophenyl)butyl]-7-(2,2-dimethylamino)ethyl-1-propyl-8-(4-sulfonoxybenzyl)xanthine

By the method of Example 2,4-sulfonoxyphenylacetic acid is reacted with 5,6-diamino-1-[4-(4-nitrophenyl)butyl]-3-propyluracil (6) to yield 3-[4-(4-nitrophenyl)butyl]-1-propyl-8-(4-sulfonoxybenzyl)xanthine. In turn, 5,6-diamino-1-[4-(4-nitrophenyl)butyl]-3-propyluracil is made by the synthetic methods of Example 1, starting with n-propyl isocyanate and 4-(4-nitrophenyl)butylamine. By the method of Example 3, 3-[4-(4-nitrophenyl)butyl]-1-propyl-8-(4-sulfonoxybenzyl)xanthine, is reacted with excess sodium carbonate, 1,2-dichloroethane and dimethylamine to yield, after aqueous work-up, 7-(2,2-dimethylamino)ethyl-3-[2-(4-nitrophenyl)butyl]-1-propyl-8-(4-sulfonoxybenzyl)xanthine. By the method of Example 4, 7-(2,2-dimethylamino)ethyl-3-[4-(4-nitrophenyl)butyl]-1-propyl-8-(4-sulfonoxybenzyl)xanthine is reduced with hydrazine hydrate or hydrogen gas in the presence of a palladium catalyst to yield 3-[4-(4-aminophenyl)butyl]-7-(2,2-dimethylamino)ethyl-1-propyl-8-(4-sulfonoxybenzyl)xanthine.

### Example 37

Pharmaceutical Formulations

(A) Tablet

| | Amount per Tablet |
|---|---|
| Active Ingredient: Compound of Formula (I) | 150 mg |
| Starch | 50 mg |
| Microcrystalline cellulose | 45 mg |
| Polyvinylpryrrolidone (as 10% solution in water) | 5 mg |
| Sodium carboxymethyl starch | 5 mg |
| Magnesium stearate | 1 mg |
| Talc | 1 mg |

The active ingredient, starch and cellulose are passed through a No. 45 mesh U.S. sieve and mixed thoroughly. The aqueous solution containing polyvinylpyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed in a tablet machine to yield tablets.

(B) Capsule

| | Amount per Capsule |
|---|---|
| Active Ingredient: Compound of Formula (I) | 150 mg |
| Starch | 24 mg |
| Microcrystalline cellulose | 24 mg |
| Magnesium stearate | 2 mg |

The active ingredient, cellulose, starch and magnesium stearate are blended, passed through a No. 45 mesh U.S. Sieve, and filed into hard gelatin capsules.

(C) Intravenous Fluid

| | Amount per bag | |
|---|---|---|
| Active Ingredient: Compound of Formula (I) | 100 | mg |
| Sterile Isotonic saline for injection | 250 | ml |

In a sterile environment, the active ingredient is dissolved in the isotonic saline and the resulting solution is passed through a 2 micron filter then filed into sterile intravenous fluid bags that are immediately sealed.

In the specification above, there have been disclosed embodiments of the invention and, although specific terms are employed, they are used in a generic and descriptive sense only and not for purposes of limitation of the scope of the invention being set forth in the following claims.

What is claimed is:

1. A diagnostic probe comprising a compound of formula (I): 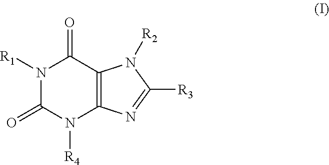
wherein;
R₁ is C₁₋₈ straight or branched alkyl optionally substituted with one or more OR₅, NR₆R₇, or halogen groups, and when the aromatic group of R₃ is other than phenyl, R₁ may also be C₁₋₈ straight or branched alkyl
wherein;
R₅ and R₆ are independently H, or C₁₋₈ straight or branched alkyl;
R₇ is H, C₁₋₈ straight or branched alkyl, or Alk₁-OH,
wherein; Alk₁ is C₁₋₈ straight or branched alkylene;
R₂ is H, C₁₋₈ alkyl, Alk₂COOH, Alk₃COOR₈, Alk₄CONR₉R₁₀, Alk₅OH, Alk₆SO₃H, Alk₇PO₃H₂, Alk₈OR₁₁, Alk₉OH, Alk₁₀NR₁₂R₁₃, or, when R₃ is (CH₂)_{q}(C₆H₄)Q, R₂ may also be Alk₁₁N(CH₃)Alk₁₂OH; and when R₃ is other than (CH₂)_{q}(C₆H₄)Q, R₂ may also be Alk₁₃NR₁₄R₁₅;
wherein;
Alk₂ through Alk₁₃ are independently C₁₋₈ straight or branched alkylene or alkenylene;
q is an integer ranging from 1 to 8;
Q is H, OH, NH₂, (CH2)ₜ OH, or R₁₃ₐCOOH, wherein t is an integer ranging from 1 to 8;
R₈ through R₁₃ are independently H or C₁₋₈ straight or branched alkyl;
R₁₃ₐ is C₁₋₈ straight or branched alkylene;
R₁₄ is H, CH₃, or (CH₂)ₚ₁CH₃;
R₁₅ is H, CH₃, (CH₂)ₚ₂CH₃ or (CH₂)ₘOH,
wherein; p1 and p2 are independently integers from 1 to 7, and m is an integer from 1 to 8;
R₃ is Alk₁₄ArR₁₆,
wherein;
Alk₁₄ is C₁₋₈ straight or branched alkylene or alkenylene;
Ar is a 5- or 6-member aromatic ring containing 0 to 4 heteroatoms selected from N, O, and S, or is a bicyclic 9- or 11-member aromatic ring containing 0 to 6 heteroatoms selected from N, O, and S;
R₁₆ is H, OH, OR_{13b}, NO₂, NH₂, CN, Alk₁₅OH, Alk₁₆NH₂, NR₁₇R₁₈, NR₁₉COR₁₉ₐ, Alk₁₇COOR_{19b}, SO₂R_{19c}, SO₃H, and PO₃H₂, or halogen;
wherein;
Alk₁₅ through Alk₁₇ are independently C₁₋₈ straight or branched alkylene or alkenylene;
R_{13b} is H, or C₁₋₈ straight or branched alkyl;
R₁₇, through R₁₉ and R₁₉ₐ through R_{19c} are independently H, an aromatic group, or C₁₋₈ straight or branched alkyl;
R₄ is 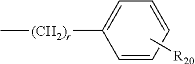
wherein;
r is an integer from 1 to 20;
R₂₀ is SO₃H, PO₃H₂, halogen, OR_{13c}, COOR_{13d}, NO₂, NR₂₁R₂₂, NR₂₃COR₂₃ₐ, Alk₁₈COOR_{19d}, SO₂R₁₉ₑ, and Alk₁₈NR₂₄R₂₅ and when the aromatic group of R₃ is other than (CH₂)_{q}(C₆H₄)Q, R₂₀ may also be H, OH, NH₂ Alk₁₉OH, or Alk₂₀NH₂, or Alk₂₁COOH;
wherein;
Alk₁₉ through Alk₂₁ are independently C₁₋₈ straight or branched alkylene or alkenylene;
R_{13c} and R_{13d} are independently C₁₋₈ straight or branched alkyl;
R_{19d} and R₁₉ₑ are independently H, an aromatic group or C₁₋₈ straight or branched alkyl;
R₂₁, through R₂₅ and R₂₃ₐ are independently H, an aromatic group or C₁₋₈ straight or branched alkyl;
with one or more radioactive or non-radioactive label moieties attached thereto.

2. The diagnostic probe according to claim 1, wherein the label moieties are connected to the compound of formula (I) through one or more spacer or chelate moieties.

3. The diagnostic probe according to claim 2, wherein each spacer or chelate moiety has functionality that bonds to one or more amine, hydroxyl, or carboxyl functionalities of the compound of formula (I).

4. The diagnostic probe according to claim 1, wherein the non-radioactive label moiety is a fluorophore label.

5. The diagnostic probe according to claim 1, wherein the non-radioactive label moiety is a chelated lanthanide.

6. The diagnostic probe according to claim 5, wherein the lanthanide is europium.

7. The diagnostic probe according to claim 1, wherein the non-radioactive label moiety is a luminescent dye.

8. The diagnostic probe according to claim 1, wherein the non-radioactive radioactive label moiety is biotin.

9. The diagnostic probe according to claim 1, wherein the non-radioactive label moiety is obelin.

10. The diagnostic probe according to claim 1, wherein the compound is labeled by the radioactive label moiety connected by a spacer component, and the spacer component has functionality, which bonds to one or more of the amine, hydroxyl, or carboxyl functionalities present on the R₁, R₂, R₃ or R₄ constituent of the compound.

11. The diagnostic probe according to claim 1, wherein the compound is labeled by the radioactive material, which is 1) a radioactive isotope selected from the group consisting of ¹⁸F, tritium, ¹¹C, ¹³C, ¹²⁵I, or ¹⁵N; or 2) a complex of a metal atom, a metal ion, or a chelating agent.

12. An imaging agent comprising a compound of formula (I): 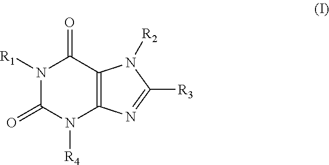
wherein;
R₁ is C₁₋₈ straight or branched alkyl optionally substituted with one or more OR₅, NR₆R₇, or halogen groups, and when the aromatic group of R₃ is other than phenyl, R₁ may also be C₁₋₈ straight or branched alkyl
wherein;
R₅ and R₆ are independently H, or C₁₋₈ straight or branched alkyl;
R₇ is H, C₁₋₈ straight or branched alkyl, or Alk₁-OH,
wherein; Alk₁ is C₁₋₈ straight or branched alkylene;
R₂ is H, C₁₋₈ alkyl, Alk₂COOH, Alk₃COOR₈, Alk₄CONR₉R₁₀, Alk₅OH, Alk₆SO₃H, Alk₇PO₃H₂, Alk₈OR₁₁, Alk₉OH, Alk₁₀NR₁₂R₁₃, or, when R₃ is (CH₂)_{q}(C₆H₄)Q, R₂ may also be Alk₁₁N(CH₃)Alk₁₂OH; and when R₃ is other than (CH₂)_{q}(C₆H₄)Q, R₂ may also be Alk₁₃NR₁₄R₁₅;
wherein;
Alk₂ through Alk₁₃ are independently C₁₋₈ straight or branched alkylene or alkenylene;
q is an integer ranging from 1 to 8;
Q is H, OH, NH₂, (CH2)ₜ OH, or R₁₃ₐCOOH, wherein t is an integer ranging from 1 to 8;
R₈ through R₁₃ are independently H or C₁₋₈ straight or branched alkyl;
R₁₃ₐ is C₁₋₈ straight or branched alkylene;
R₁₄ is H, CH₃, or (CH₂)ₚ₁CH₃;
R₁₅ is H, CH₃, (CH₂)ₚ₂CH₃ or (CH₂)ₘOH,
wherein; p1 and p2 are independently integers from 1 to 7, and m is an integer from 1 to 8;
R₃ is Alk₁₄ArR₁₆,
wherein;
Alk₁₄ is C₁₋₈ straight or branched alkylene or alkenylene;
Ar is a 5- or 6-member aromatic ring containing 0 to 4 heteroatoms selected from N, O, and S, or is a bicyclic 9- or 11-member aromatic ring containing 0 to 6 heteroatoms selected from N, O, and S;
R₁₆ is H, OH, OR_{13b}, NO₂, NH₂, CN, Alk₁₅OH, Alk₁₆NH₂, NR₁₇R₁₈, NR₁₉COR₁₉ₐ, Alk₁₇COOR_{19b}, SO₂R_{19c}, SO₃H, and PO₃H₂, or halogen;
wherein;
Alk₁₅ through Alk₁₇ are independently C₁₋₈ straight or branched alkylene or alkenylene;
R_{13b} is H, or C₁₋₈ straight or branched alkyl;
R₁₇, through R₁₉ and R₁₉ₐ through R_{19c} are independently H, an aromatic group, or C₁₋₈ straight or branched alkyl;
R₄ is 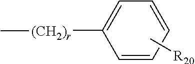
wherein;
r is an integer from 1 to 20;
R₂₀ is SO₃H, PO₃H₂, halogen, OR_{13c}, COOR_{13d}, NO₂, NR₂₁R₂₂, NR₂₃COR₂₃ₐ, Alk₁₈COOR_{19d}, SO₂R₁₉ₑ, and Alk₁₈NR₂₄R₂₅ and when the aromatic group of R₃ is other than (CH₂)_{q}(C₆H₄)Q, R₂₀ may also be H, OH, NH₂ Alk₁₉OH, or Alk₂₀NH₂, or Alk₂₁COOH;
wherein;
Alk₁₉ through Alk₂₁ are independently C₁₋₈ straight or branched alkylene or alkenylene;
R_{13c} and R_{13d} are independently C₁₋₈ straight or branched alkyl;
R_{19d} and R₁₉ₑ are independently H, an aromatic group or C₁₋₈ straight or branched alkyl;
R₂₁, through R₂₅ and R₂₃ₐ are independently H, an aromatic group or C₁₋₈ straight or branched alkyl;
having one or more radioactive, nuclear spin, or radioactive and nuclear spin; or non-radioactive label marker atoms attached thereto.

13. The imaging agent of according to claim 12, wherein the marker atoms are connected to compound of formula (I) through one or more spacer or chelate moieties.

14. The imaging agent of according to claim 13, wherein the marker atom is a radioactive isotope.

15. The imaging agent of according to claim 14, wherein the radioactive isotope is ¹⁸F, ¹¹C, ¹⁵N, ¹²⁵I, or tritium.

16. The imaging agent of according to claim 12, wherein the nuclear spin marker atom is ¹⁹F.

17. A PET imaging agent comprising a compound of formula (I): 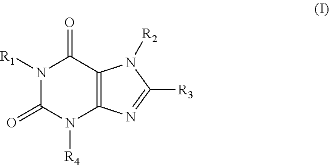
wherein;
R₁ is C₁₋₈ straight or branched alkyl optionally substituted with one or more OR₅, NR₆R₇, or halogen groups, and when the aromatic group of R₃ is other than phenyl, R₁ may also be C₁₋₈ straight or branched alkyl
wherein;
R₅ and R₆ are independently H, or C₁₋₈ straight or branched alkyl;
R₇ is H, C₁₋₈ straight or branched alkyl, or Alk₁-OH,
wherein; Alk₁ is C₁₋₈ straight or branched alkylene;
R₂ is H, C₁₋₈ alkyl, Alk₂COOH, Alk₃COOR₈, Alk₄CONR₉R₁₀, Alk₅OH, Alk₆SO₃H, Alk₇PO₃H₂, Alk₈OR₁₁, Alk₉OH, Alk₁₀NR₁₂R₁₃, or, when R₃ is (CH₂)_{q}(C₆H₄)Q, R₂ may also be Alk₁₁N(CH₃)Alk₁₂OH; and when R₃ is other than (CH₂)_{q}(C₆H₄)Q, R₂ may also be Alk₁₃NR₁₄R₁₅;
wherein;
Alk₂ through Alk₁₃ are independently C₁₋₈ straight or branched alkylene or alkenylene;
q is an integer ranging from 1 to 8;
Q is H, OH, NH₂, (CH2)ₜ OH, or R₁₃ₐCOOH, wherein t is an integer ranging from 1 to 8;
R₈ through R₁₃ are independently H or C₁₋₈ straight or branched alkyl;
R₁₃ₐ is C₁₋₈ straight or branched alkylene;
R₁₄ is H, CH₃, or (CH₂)ₚ₁CH₃;
R₁₅ is H, CH₃, (CH₂)ₚ₂CH₃ or (CH₂)ₘOH,
wherein; p1 and p2 are independently integers from 1 to 7, and m is an integer from 1 to 8;
R₃ is Alk₁₄ArR₁₆,
wherein;
Alk₁₄ is C₁₋₈ straight or branched alkylene or alkenylene;
Ar is a 5- or 6-member aromatic ring containing 0 to 4 heteroatoms selected from N, O, and S, or is a bicyclic 9- or 11-member aromatic ring containing 0 to 6 heteroatoms selected from N, O, and S;
R₁₆ is H, OH, OR_{13b}, NO₂, NH₂, CN, Alk₁₅OH, Alk₁₆NH₂, NR₁₇R₁₈, NR₁₉COR₁₉ₐ, Alk₁₇COOR_{19b}, SO₂R_{19c}, SO₃H, and PO₃H₂, or halogen;
wherein;
Alk₁₅ through Alk₁₇ are independently C₁₋₈ straight or branched alkylene or alkenylene;
R_{13b} is H, or C₁₋₈ straight or branched alkyl;
R₁₇, through R₁₉ and R₁₉ₐ through R_{19c} are independently H, an aromatic group, or C₁₋₈ straight or branched alkyl;
R₄ is 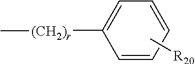
wherein;
r is an integer from 1 to 20;
R₂₀ is SO₃H, PO₃H₂, halogen, OR_{13c}, COOR_{13d}, NO₂, NR₂₁R₂₂, NR₂₃COR₂₃ₐ, Alk₁₈COOR_{19d}, SO₂R₁₉ₑ, and Alk₁₈NR₂₄R₂₅ and when the aromatic group of R₃ is other than (CH₂)_{q}(C₆H₄)Q, R₂₀ may also be H, OH, NH₂ Alk₁₉OH, or Alk₂₀NH₂, or Alk₂₁COOH;
wherein;
Alk₁₀ through Alk₂₁ are independently C₁₋₈ straight or branched alkylene or alkenylene;
R_{13c} and R_{13d} are independently C₁₋₈ straight or branched alkyl;
R_{19d} and R₁₉ₑ are independently H, an aromatic group or C₁₋₈ straight or branched alkyl;
R₂₁, through R₂₅ and R₂₃ₐ are independently H, an aromatic group or C₁₋₈ straight or branched alkyl;
having one or more labeled marker atoms.
